SEQUENCE LISTING (2 sequences)

SEQ ID NO: 1 - DNA, 19 bp, Artificial Sequence
  Oligonucleotide
tccatgacgttccgatgct

SEQ ID NO: 2 - protein, 235 aa, Homo sapien
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

(19) United States
(12) Patent Application Publication
(10) Pub. No.: US 2010/0016240 A1
(43) Pub. Date: Jan. 21, 2010

(54) FACILITATING CELLS AND DIABETES AND METHODS RELATED THERETO

(76) Inventor: Suzanne T. Ildstad, Prospect, KY (US)

Correspondence Address: FISH & RICHARDSON P.C., PO BOX 1022, MINNEAPOLIS, MN, 55440-1022, US

(21) Appl. No.: 12/485,605

(22) Filed: Jun. 16, 2009

Related U.S. Application Data
(60) Provisional application No. 61/061,860, filed on Jun. 16, 2008.

(51) Int. Cl. A61K 38/08; G01N 33/53; A61P 3/10

(52) U.S. Cl. 514/15; 435/7.21; 514/17

(57) ABSTRACT

This disclosure describes methods of screening for compounds that increase the number of p-preDCs and/or Tregs.

### CROSS REFERENCE TO RELATED APPLICATIONS

This application claims benefit under 35 U.S.C. 119(e) to U.S. Provisional Application No. 61/061,860 filed Jun. 16, 2008.

### BACKGROUND

A CD8⁺/TCR⁻ facilitating cell (FC) population was previously identified in mouse bone marrow (BM) that facilitates hematopoietic stem cell (HSC) engraftment across major histocompatibility complex (MHC) barriers without causing graft-versus-host disease (GVHD). FCs are a heterogeneous cell population, with the predominant subpopulation resembling B220⁺/CD11c⁺/CD11b⁻ plasmacytoid precursor dendritic cells (p-preDCs). P-preDC FCs display characteristic plasmacytoid morphology and produce IFN-α, TNF-α and other cytokines in response to CpG-ODN. P-preDC FCs also have the capacity to differentiate into mature DC by up-regulating MHC class II, CD86, and CD80 activation markers. Removal of p-pre DC FC completely abrogates facilitation, suggesting that p-preDC FC play a critical role in facilitation.

NOD mice develop spontaneous autoimmune diabetes due to defects in both peripheral and central tolerance mechanisms. Several regulatory defects have been described in NOD mice, including islet-reactive T cells that escape deletion, impaired generation of regulatory T cells (T_{reg}), inhibitory cytokines, aberrant professional APC function, and low levels of NK cell activity. B cells also contribute to the development of diabetes in NOD in their role as professional APC. The role of p-preDCs in pathogenesis of autoimmune disease has been studied. Several groups reported abnormalities in DC phenotype and function in human type I diabetes and NOD mice. An understanding of which specific DC subsets play a critical role on maintenance in self-tolerance and prevention of diabetes may allow novel cell-based therapies to be utilized in the clinic for disease prevention.

### SUMMARY

The invention provides a correlation between diabetes or other autoimmune diseases and facilitating cells. Thus, methods of evaluating an individual for the likelihood of developing diabetes are provided, as are methods of treating an individual having diabetes. In addition, this disclosure describes methods of screening for compounds that can stimulate the production of p-preDCs and/or Tregs.

In one aspect, methods of screening for a compound that stimulates the production of p-preDC cells are provided. Such methods typically include contacting facilitating cells (FCs) with a test compound; and determining whether or not the test compound increases the number of p-preDC cells. Generally, an increase in the number of p-preDC cells is indicative of a compound that stimulates the production of p-preDC cells. The compound can be a polypeptide, a small molecule, and a chemical. In some embodiments, the number of p-preDC cells is determined using FACS.

In another aspect, methods of screening for a compound that stimulates the production of antigen-specific Treg cells are provided. Such methods typically include contacting facilitating cells (FCs) with a test compound; and determining whether or not the test compound increases the number of antigen-specific Treg cells. Generally, an increase in the number of antigen-specific Treg cells is indicative of a compound that stimulates the production of antigen-specific Treg cells. The compound can be a polypeptide, a small molecule, and a chemical. In some embodiments, the number of antigen-specific Treg cells is determined using FACS.

In another aspect, methods of treating an individual having diabetes are provided. Such methods typically include administering a compound to the individual that increases the production of p-preDCs and/or Tregs in the individual. In one embodiment, the compound is a polypeptide. Representative polypeptides have the sequence shown in SEQ ID NO:2 or have at least 90% sequence identity (e.g., at least 95% sequence identity, at least 99% sequence identity) to the sequence shown in SEQ ID NO:2.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

### Section A

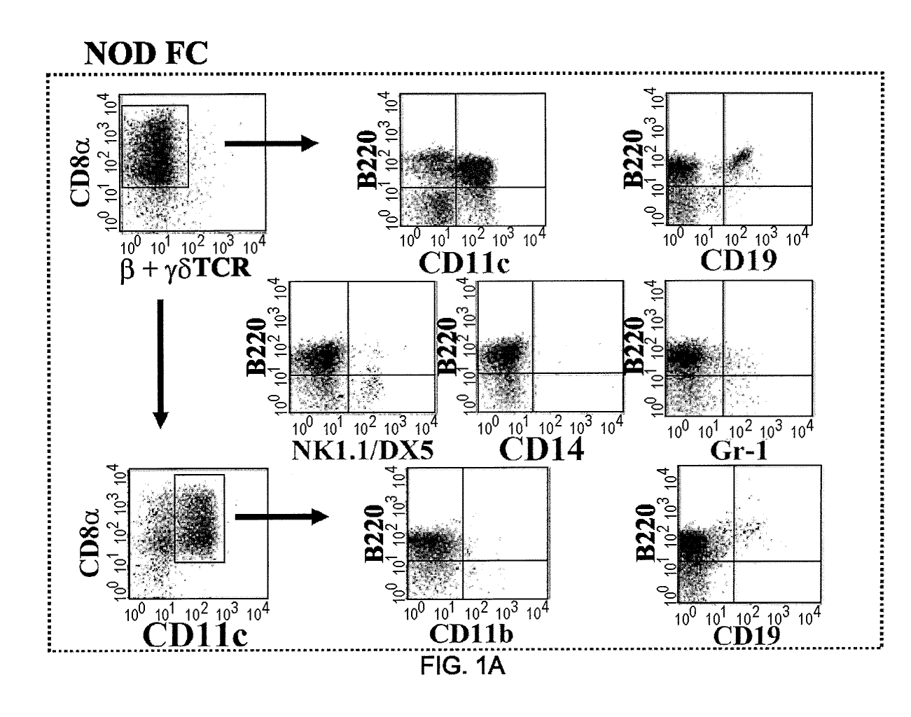

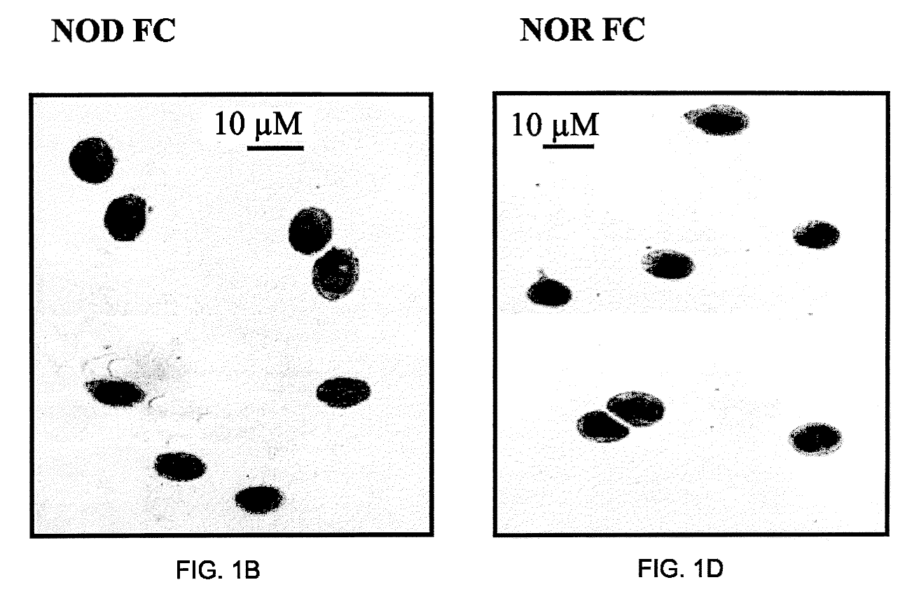

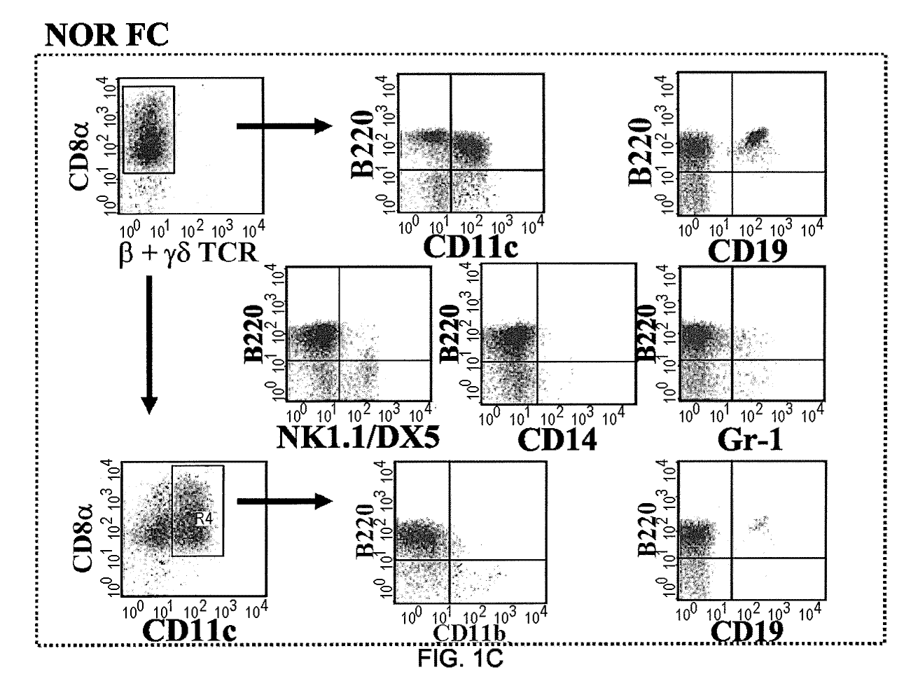

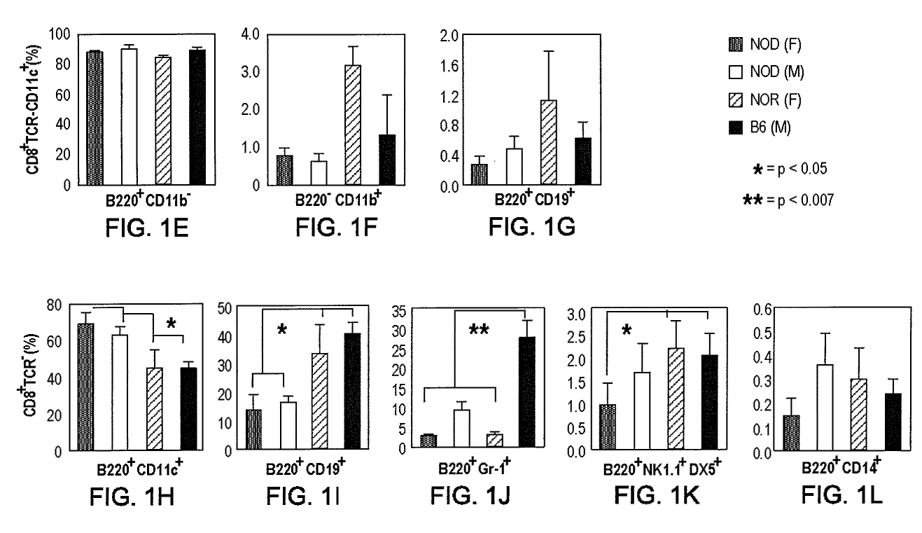

FIG. 1. NOD FC total are a heterogeneous population. (A) and (C) CD8⁺/TCR⁻ FC were sorted from NOD BM, blocked using the anti-Fc receptor Ab, and stained with anti-B220, anti-CD19, anti-CD11b, anti-NK1.1, anti-DX5, anti-Gr-1, and anti-CD14 mAbs. Flow cytometric profiles are representative of at least three experiments in NOD or NOR mice. (B) and (D) Morphology of sorted NOD or NOR CD8⁺/TCR⁻ FC were examined after Wright-Giemsa staining with optical microscopy. (E) through (L) comparison of phenotype of FC from NOD, NOR and B6 BM. Represented is the mean±SD of three independent experiments. *=P<0.05; **=P<0.007.

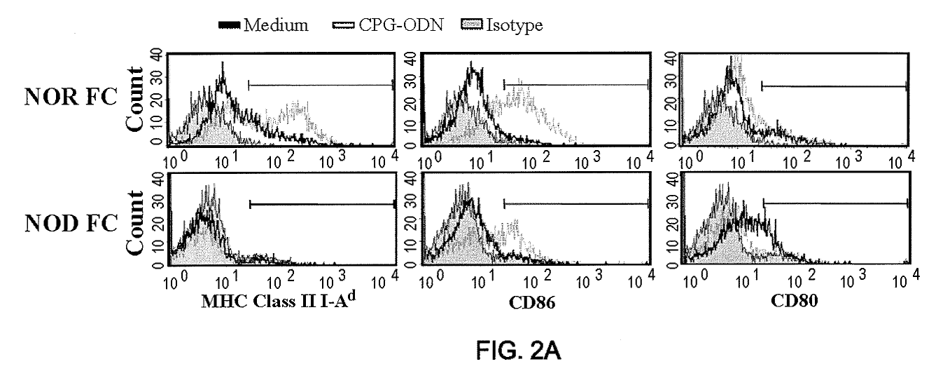

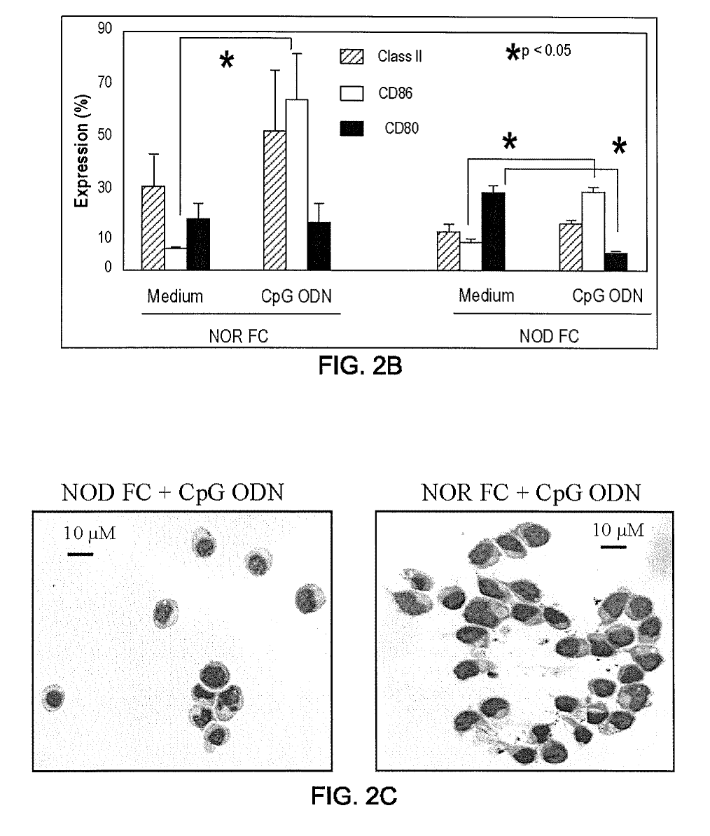

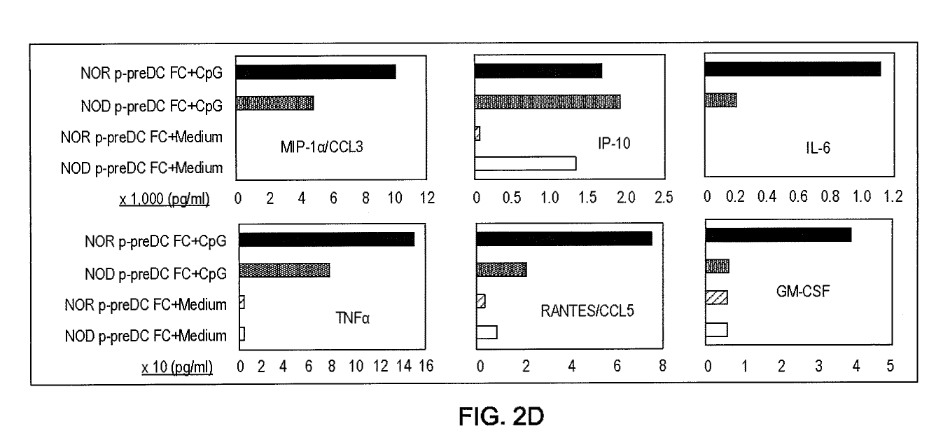

FIG. 2. Expression of activation markers on FC. (A) Sorted CD8⁺/TCR⁻ FC (100,000) from NOD or NOR BM were cultured with medium or CpG-ODN for 18 h and stained with anti-MHC Class II I-A^{d}, CD86 or CD80 FITC labeled mAb, or isotype controls. The data shown are representative of three experiments. (B) Level of expression of activation markers on FC with or without CpG-ODN stimulation. The results are percent of CD8⁺/TCR⁻ FC total from 3 separate experiments. (C) The morphology of FC was examined using Wright-Giemsa staining under optical microscopy after CpG-ODN stimulation. (D) P-preDC FC were cultured with CpG-ODN or medium only. Culture cell-free supernatants were collected after 18 h and MIP-1α/CCL3, MCP-1/CCL2, RANTES/CCL5, IP-10, IL-6, GM-CSF, and TNF-α were measured by LINCO plex multiplex immunoassay. Data showed an average of two separate experiments.

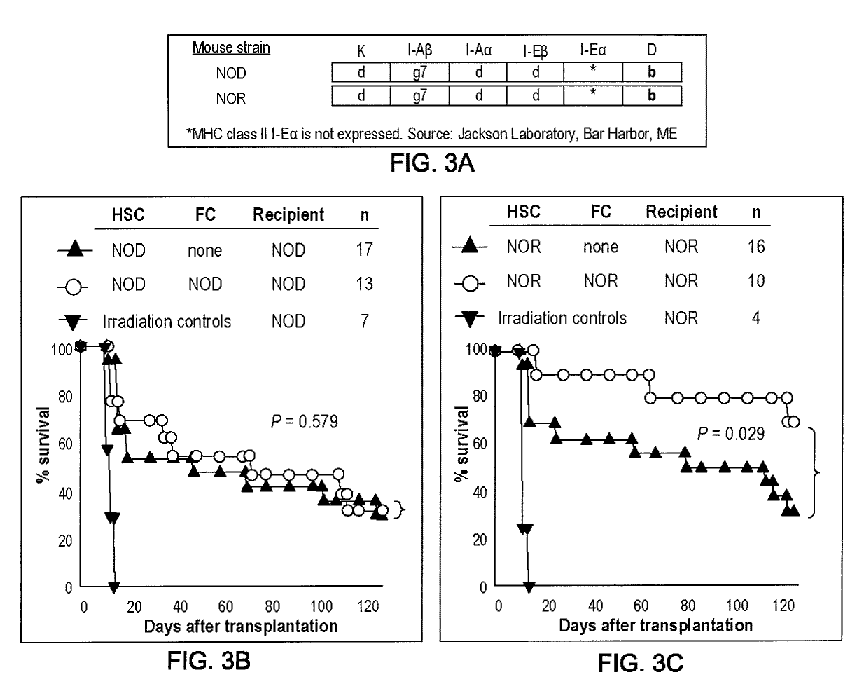

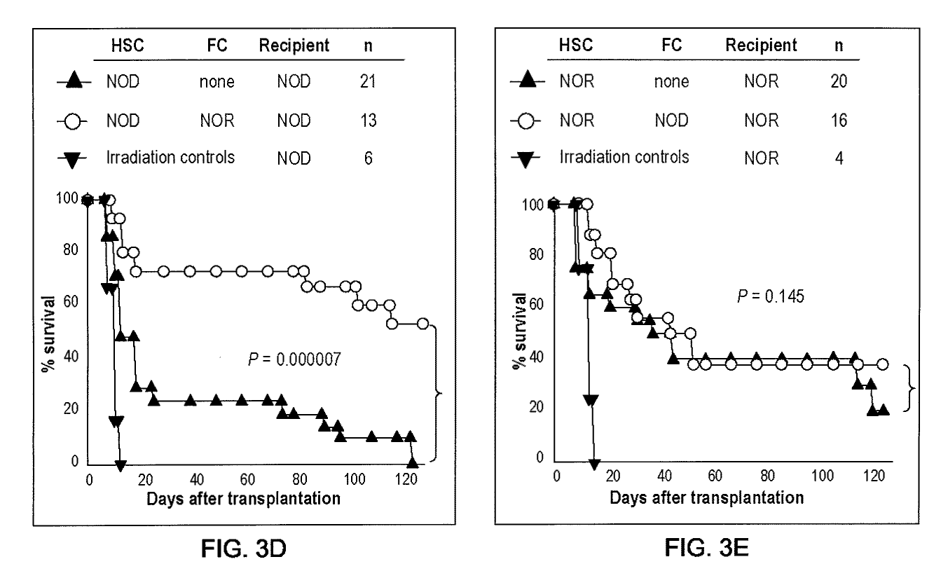

FIG. 3. NOD FC are functionally impaired in vivo. HSC (500) and FC (30,000) were sorted from donor marrow and transplanted into syngeneic recipients. HSC and FC were mixed prior to injection. A. Haplotype pedigree of NOD and NOR mice. (B) Survival of NOD recipients of NOD HSC with or without NOD FC. (C) Survival of NOR recipients of HSC and FC or HSC alone from NOR donors. (D) Survival of NOD recipients of HSC from NOD mice with or without additional FC from NOR mice. (E) Survival of NOR recipients of HSC and FC or HSC alone, HSC from NOR mice, and FC from NOD mice. Results are from 3 to 5 separate transplant experiments for each experimental group.

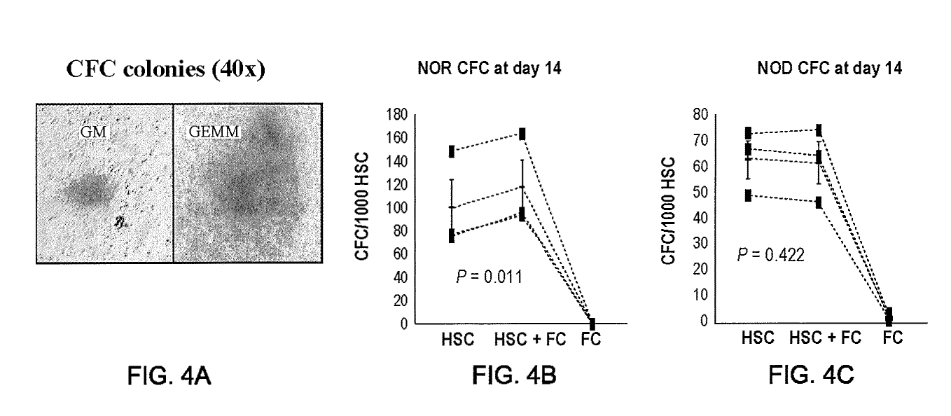

FIG. 4. NOD FC fail to promote HSC colony formation in vitro. CFC assays were performed on sorted HSC plus FC from NOD or NOR mice. (A) Representative appearance of colonies at 14 days. (B) HSC (2000) and FC (4000) were sorted from BM of NOR mice, results are expressed as CFC frequency per 1000 HSC from 3 different experiments. (C) HSC and FC from NOD mice, data represent 3 different experiments. Each rectangle (▪) represents one individual sample. The dotted lines ( - - - ) link samples from the same experiments. Averaged data from 3 experiments are presented as mean±SE.

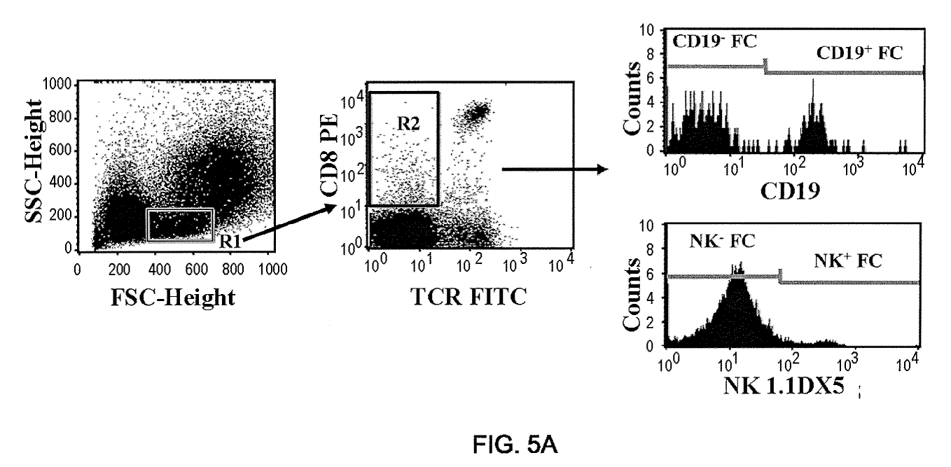

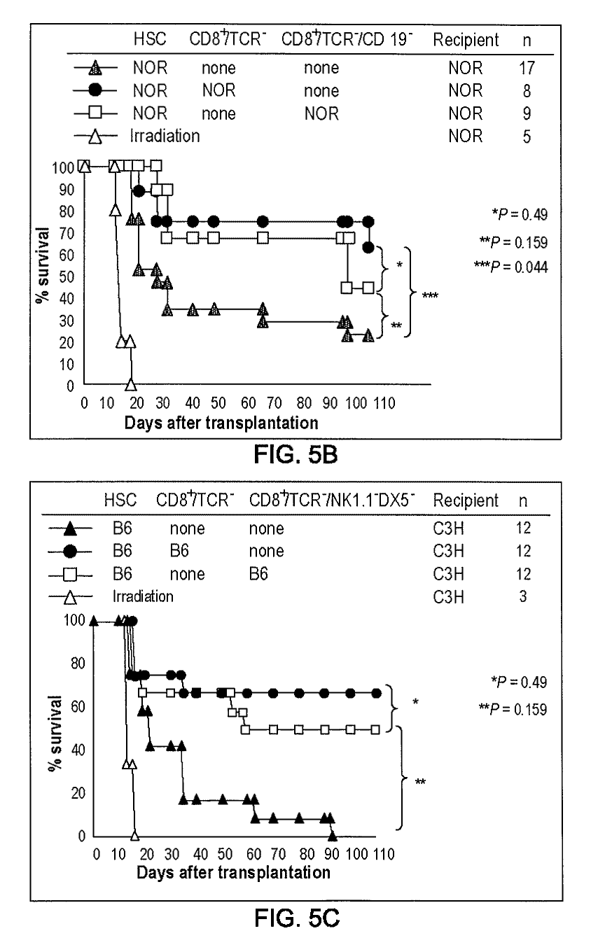

FIG. 5. Removal of CD19⁺ or NK1.1⁺DX5⁺ FC subpopulations did not significantly affect facilitation. (A) BM cells were stained with anti-CD8 PE or APC, anti-TCR-β FITC, anti-TCR-γδ FITC and anti-CD19 APC, or anti-NK1.1DX5 PE, and gated R2 for CD8⁺/TCR⁻ FC (middle panel), and CD8⁺/TCR⁻CD19⁻ or CD8⁺/TCR⁻NK1.1⁻DX5⁻ subpopulation was sorted (left panel). (B) Survival of syngeneic recipients transplanted with HSC and CD8⁺/TCR⁻ FC or CD8⁺/TCR⁻CD19⁻. NOR recipients were conditioned with 950 cGy TBI and transplanted with 500 HSC alone or in combination with 30,000 CD8⁺/TCR⁻FC (HSC+FC group), CD8⁺/TCR⁻ CD19⁻ (HSC+CD8⁺/TCR⁻CD19⁻ group). (C) Survival of recipients transplanted with HSC plus CD8⁺/TCR⁻ FC or CD8⁺/TCR⁻NK1.1⁻DX5⁻ in allogeneic model (B6→C3H). Results are from 3 to 4 separate transplant experiments.

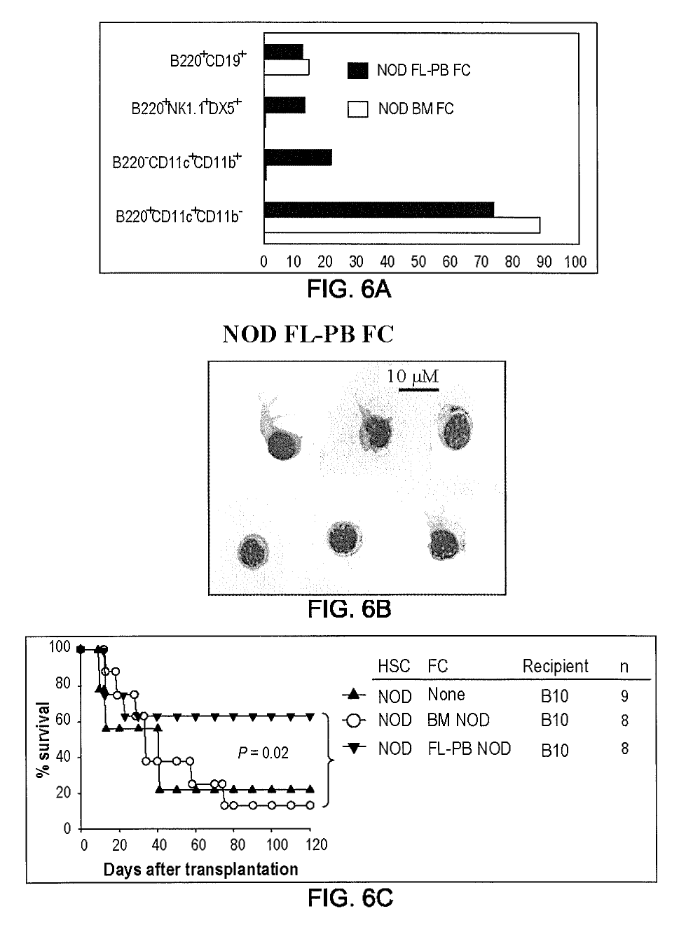

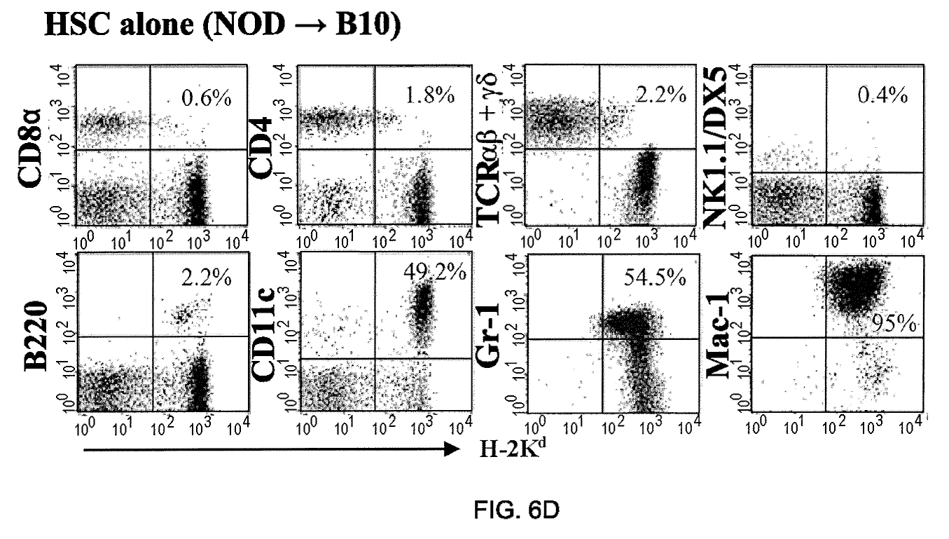

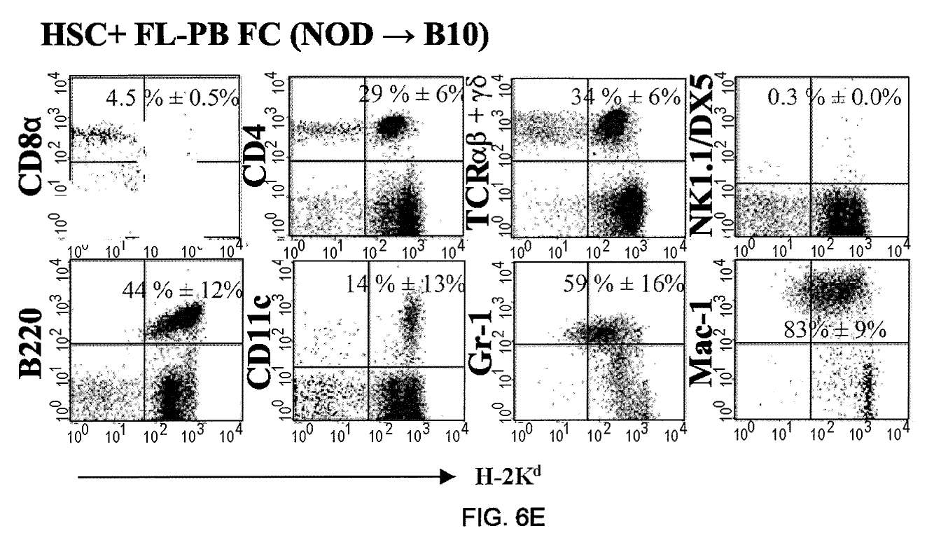

FIG. 6. FL-mobilized PB FC facilitate HSC engraftment in allogeneic recipients. Recombinant human FL (expressed from CHO cells) (kindly provided by Amgen; Thousand Oaks, Calif.) was diluted in 0.1% mouse serum albumin (MSA; Sigma, St Louis, Mo.) at a concentration of 100 μg/ml. Donor NOD female mice were injected with 10 μg of FL once daily subcutaneously from day 0 to day 9. Control mice received saline injections. (A) Flow cytometric analysis of subpopulations in sorted FL-mobilized PB FC. (B) Sorted FL-PB FC were examined after Wright-Giemsa staining by optical microscopy. (C) Survival of recipients of HSC plus FL-mobilized PB FC in allogeneic model (NOD→B10). B10 recipients were conditioned with 950 cGy TBI and transplanted with 10,000 HSC from untreated NOD donors either alone or mixed with 30,000 purified FC from untreated NOD bone marrow, or from FL-mobilized PB FC from NOD mice. (D) and (E) Multilineage typing of representative B10 recipients of NOD HSC alone or recipients of NOD HSC plus FL-PB FC. Multilineage data are from PB 3 months after transplantation and analyzed based on the lymphoid and myeloid gate. Data shown are from one representative recipient. A total of 3 to 5 recipients were analyzed per group.

### Section B

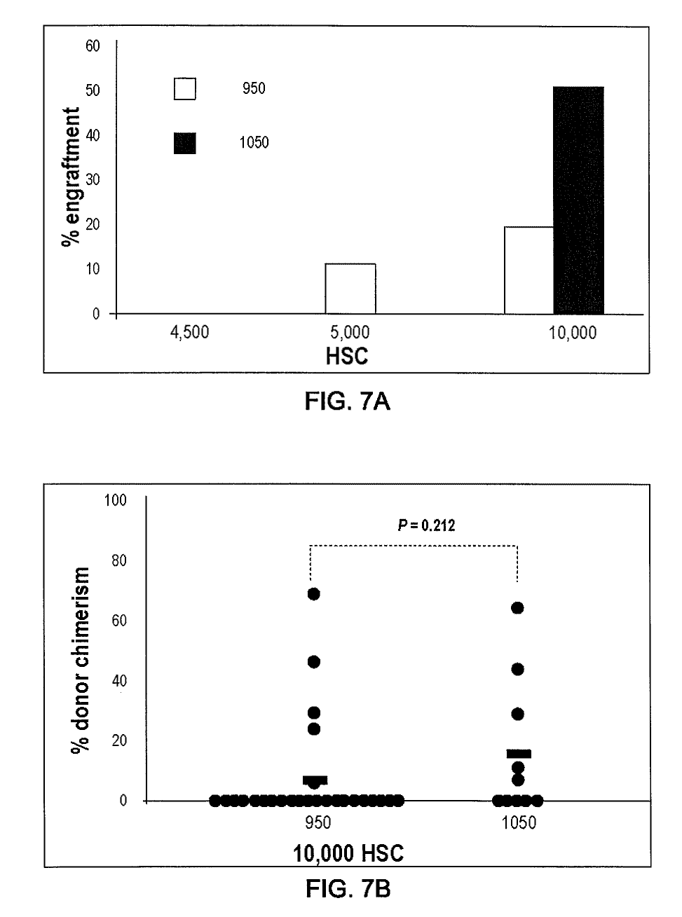

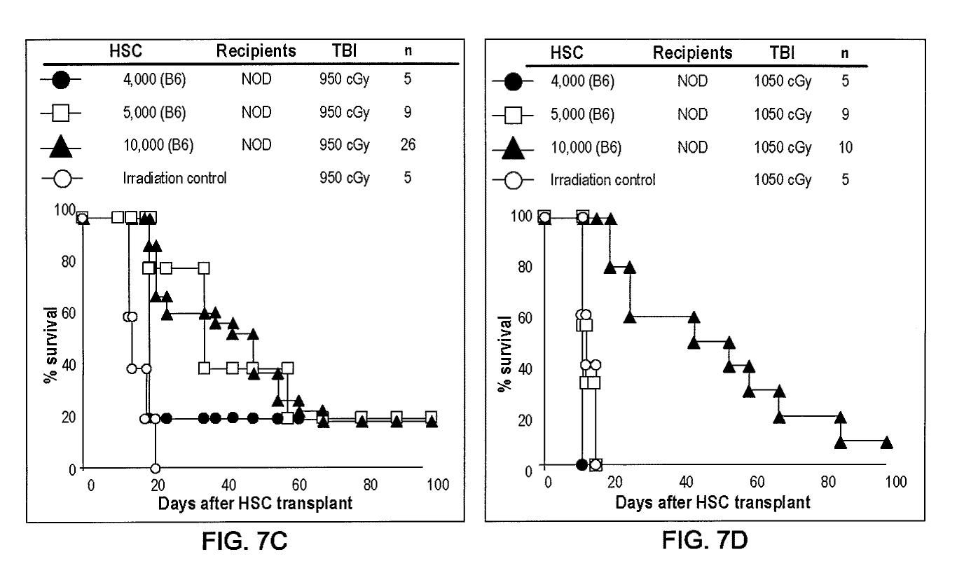

FIG. 7. Graphs of HSC and TBI dose titration. (A) Percent engraftment of NOD mice given 4000, 5000, or 10,000 B6 HSC and conditioned with 950 cGy or 1050 cGy TBI. (B) Percent donor chimerism in NOD recipient mice. (C) and (D) Survival of recipients conditioned with 950 cGy or 1050 cGy TBI and transplanted with various HSC doses in an allogeneic model (B6→NOD). Results are from 3-5 separate transplant experiments.

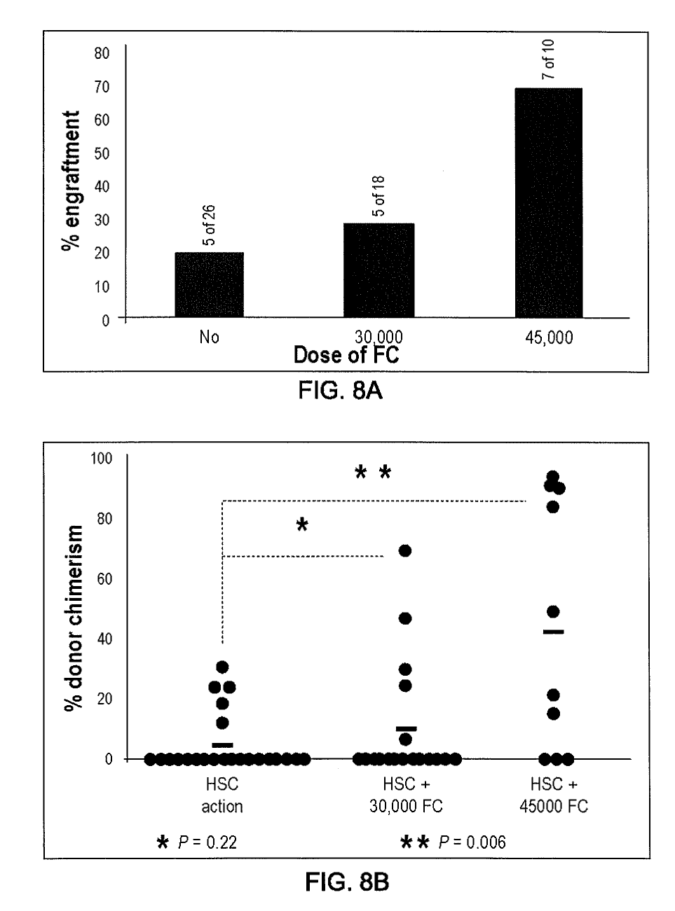

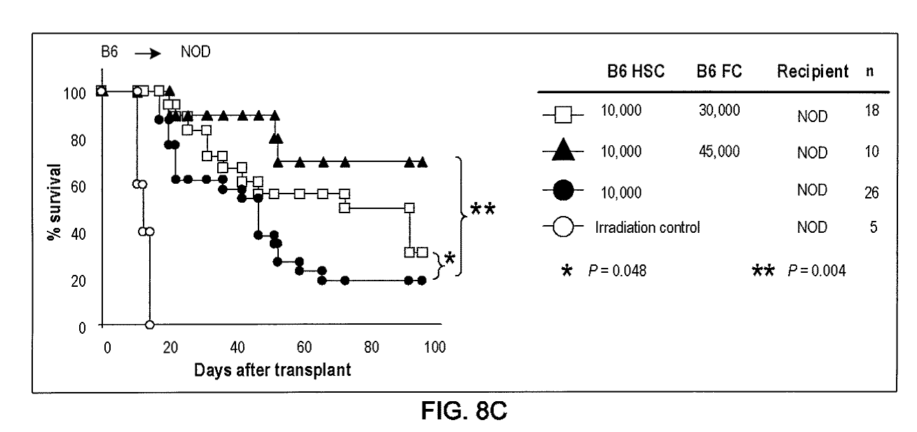

FIG. 8. Graphs demonstrating that CD8⁺/TCR⁻ FCs enhance HSC engraftment in NOD mice (B6→NOD). The ability of B6 FC to promote B6 HSC engraftment in allogeneic NOD recipients and long-term survival was evaluated. (A) Percent engraftment of NOD mice that received 10,000 B6 HSC plus 30,000 B6 FC or 45,000 B6 FC. (B) Percent donor chimerism in NOD recipient mice. Dot plots represent percent donor chimerism in individual animal. Lines indicate median percentages (*, P=0.22; **, P=0.006). (C) Survival of NOD recipients conditioned with 950 cGy TBI and transplanted with 10,000 B6 HSC with 30,000 FC (□), 10,000 B6 HSC with 45,000 B6 FC (▴), or 10,000 B6 HSC alone (). Results are shown for survival data from 3 to 5 separate transplant experiments.

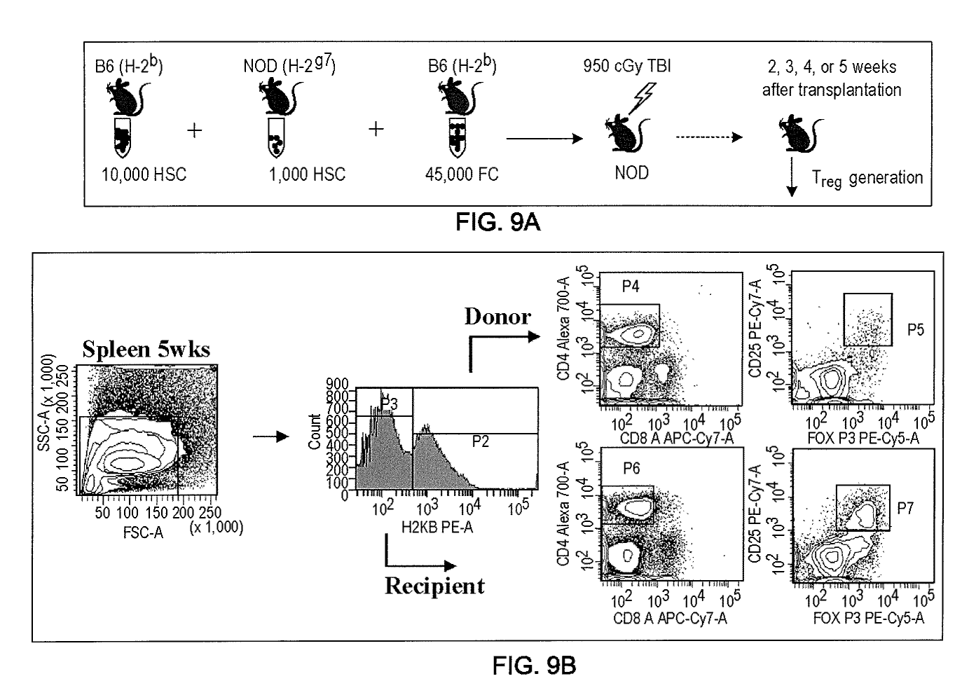

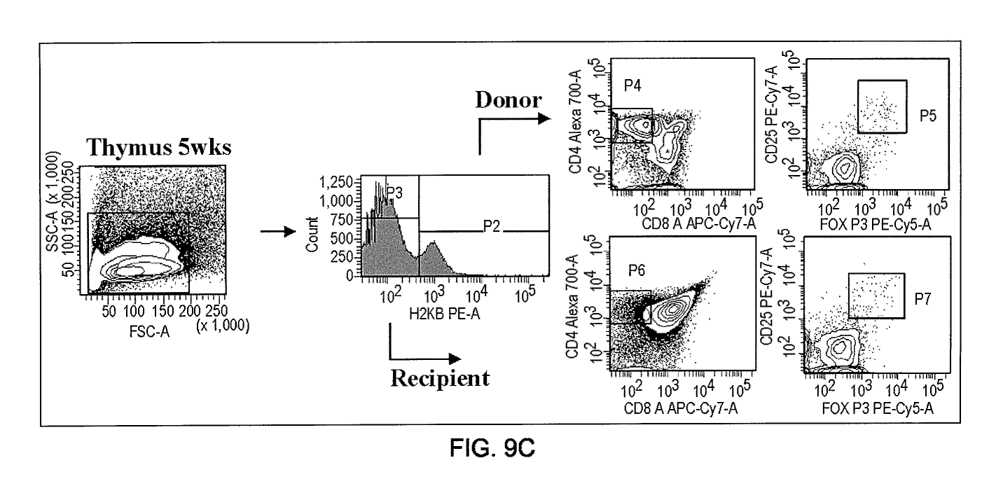

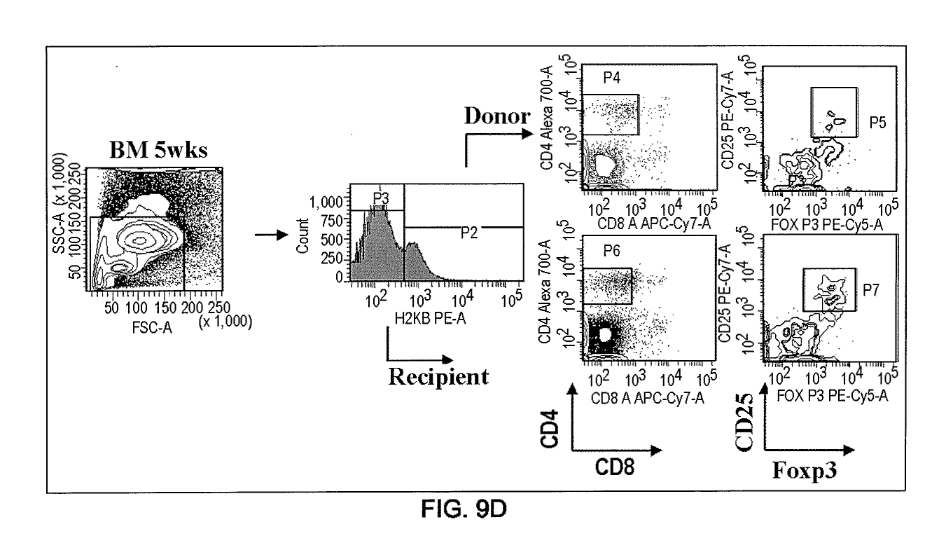

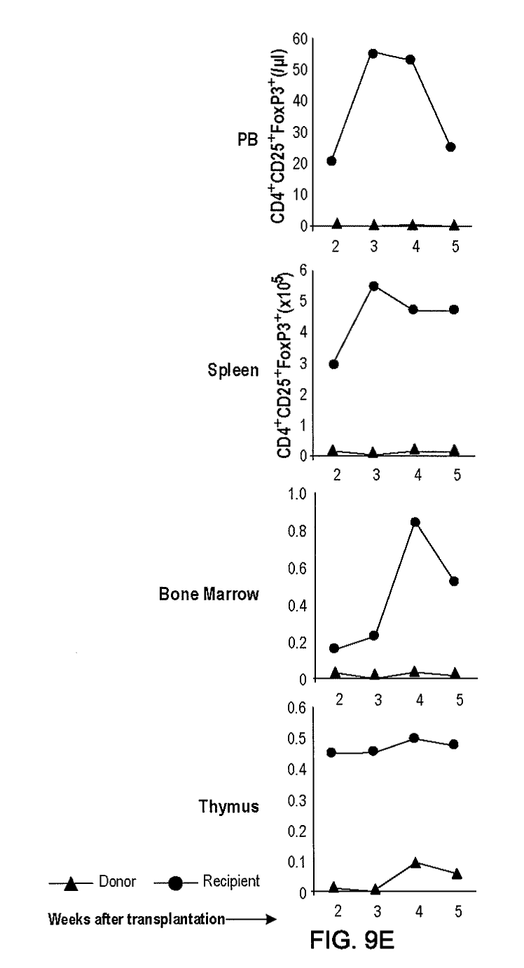

FIG. 9. Graphs demonstrating that FCs induce T_{reg} generation in vivo. Purified B6 HSC and NOD HSC with B6 FC were administered to ablatively conditioned NOD recipients and T_{reg} generation was evaluated. (A) Experimental design for the induction of in vivo T_{reg} generation. (B-D) Representative analysis of donor or recipient CD8⁻/CD4⁺/CD25⁺/FoxP3⁺ T_{reg} in chimeric spleen, thymus, bone marrow at 5 weeks after transplantation. (E) Kinetics of absolute number of donor or recipient T_{reg} in chimeric spleen, thymus, PB and bone marrow at 2, 3, 4, and 5 weeks after transplantation.

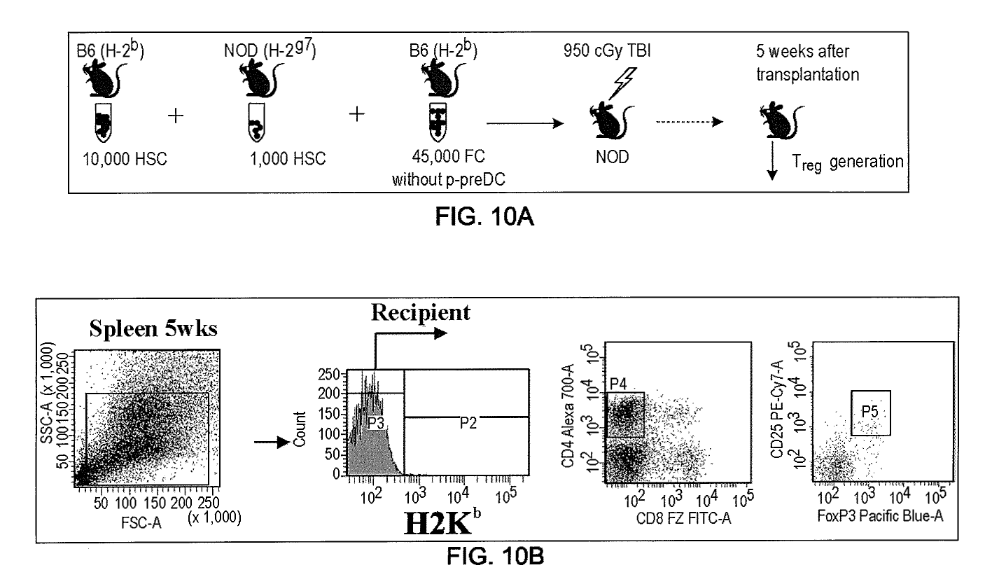

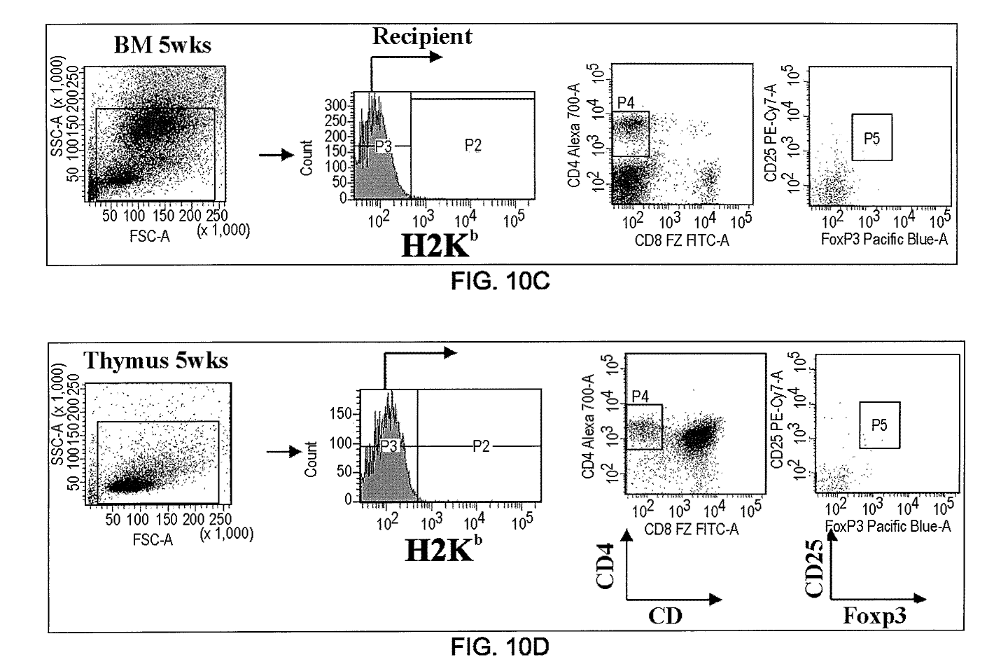

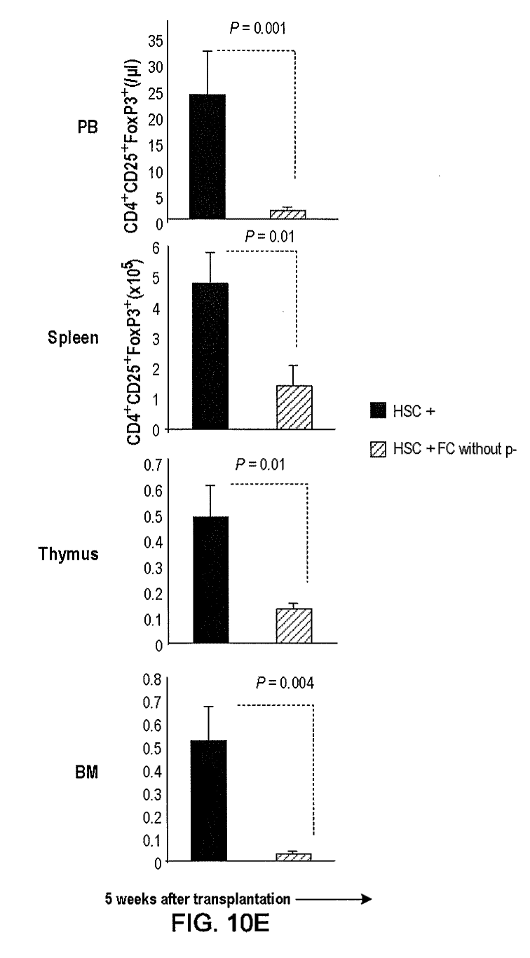

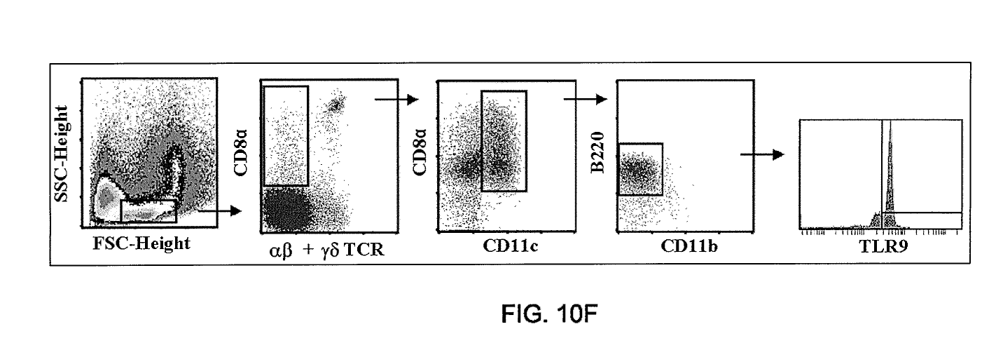

FIG. 10. Data demonstrating that the removal of p-preDC from FCs abrogated the induction of chimeric T_{reg} generation. (A) Sorted 45,000 FC, from which the p-preDC subpopulation had been removed, were transplanted with 10,000 B6 HSC plus 1,000 NOD HSC into conditioned NOD recipients. (B-D) Representative analysis of recipient CD8⁻/CD4⁺/CD25⁺/FoxP3⁺ T_{reg} in spleen, thymus, bone marrow at 5 weeks after transplantation. (E) The absolute number of recipient derived T_{reg} in PB, spleen, thymus, and BM from mice that received of HSC+FC [symbol] and HSC+FC without p-preDC (▪) 5 weeks after transplantation. (F) TLR 9 expression on p-preDC FC. CD8⁺/TCR⁻ FC were sorted from bone marrow from B6 mice. Sorted FC were stained with anti-B220, anti-CD11c, anti-CD11b, and anti-TLR 9 (clone M9.D6 from eBioscience, San Diego, Calif.) mAbs. Data showed that FC subpopulation consisted mostly of B220+CD11c+CD11b− p-preDC and this subpopulation highly expressed TLR-9.

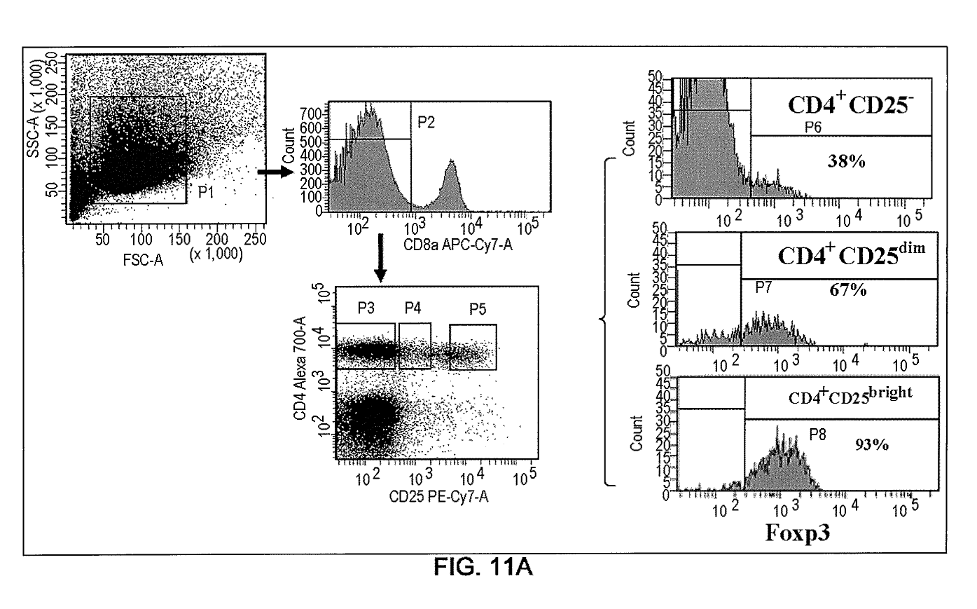

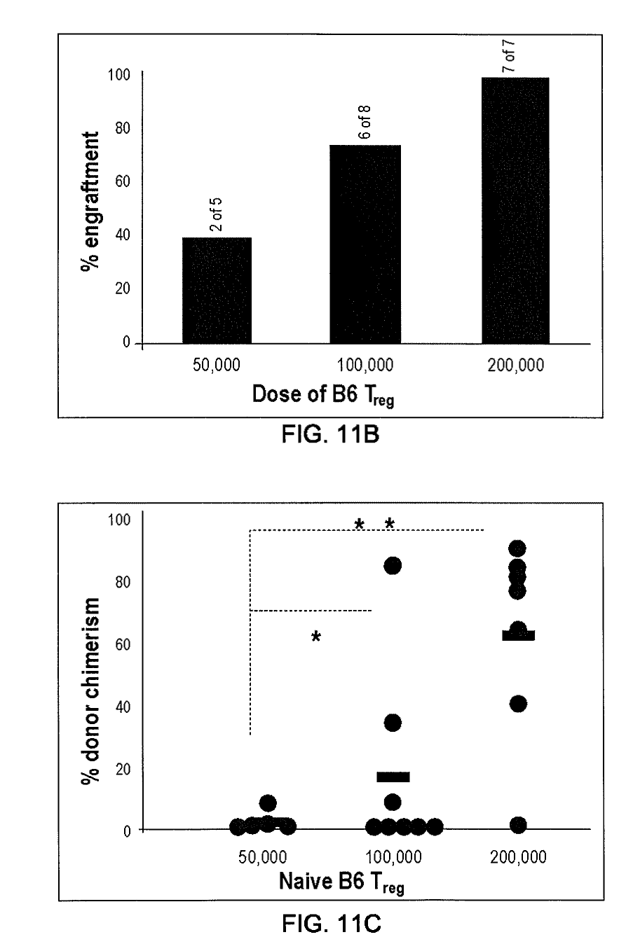

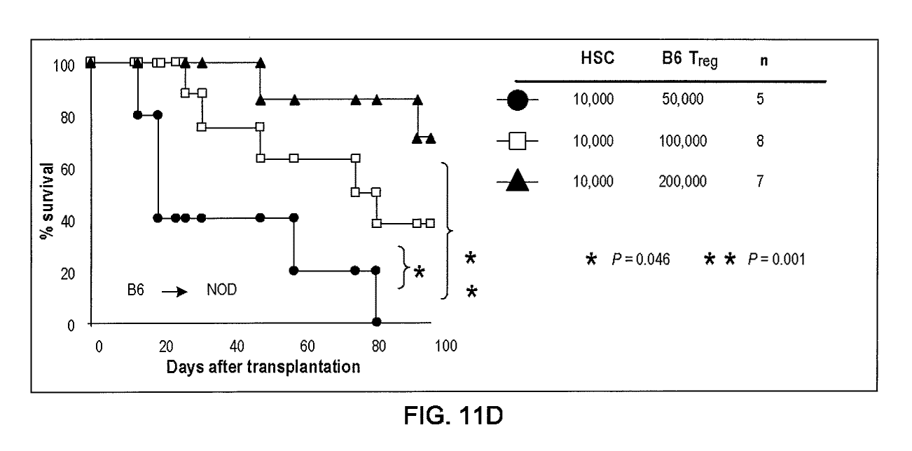

FIG. 11. Data showing that B6 T_{reg} enhance engraftment of HSC in a cell-dose dependent manner. CD8⁻/CD4⁺/CD25^{bright} T_{reg} were sorted from naïve B6 spleens. Purified B6 T_{reg} plus B6 HSC were transplanted into NOD recipients conditioned with 950 cGy TBI. (A) Splenocytes were stained (CD8⁻/CD4⁺/CD25^{bright}) and gated for CD8⁻ (upper middle panel) and CD4⁺/CD25⁻, CD4⁺/CD25^{dim} or CD4⁺/CD25^{bright} (lower middle panel). The level of FoxP3 expression in these cell fractions was analyzed (right panel). (B) Percent engraftment in NOD recipients given 10,000 B6 HSC with 50,000, 100,000 or 200,000 T_{reg} from spleens of naïve B6 mice. (C) Percent donor chimerism in NOD recipients. (D) Survival of NOD recipients conditioned 950 cGy TBI and given 10,000 B6 HSC with 50,000 B6 T_{reg} (), with 100,000 B6 T_{reg} [symbol] , or plus 200,000 B6 T_{reg} (▴). Results are from 3 separate transplant experiments.

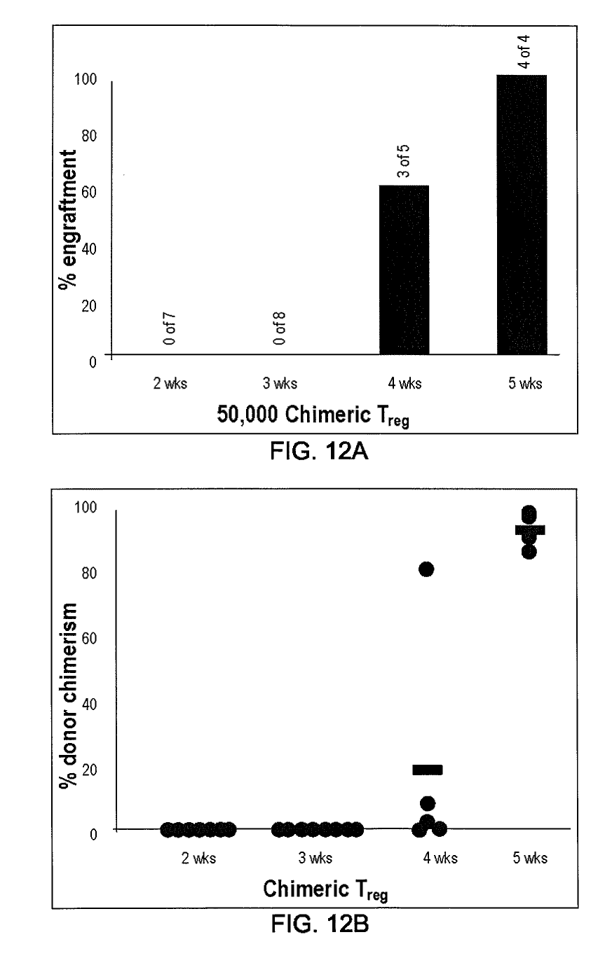

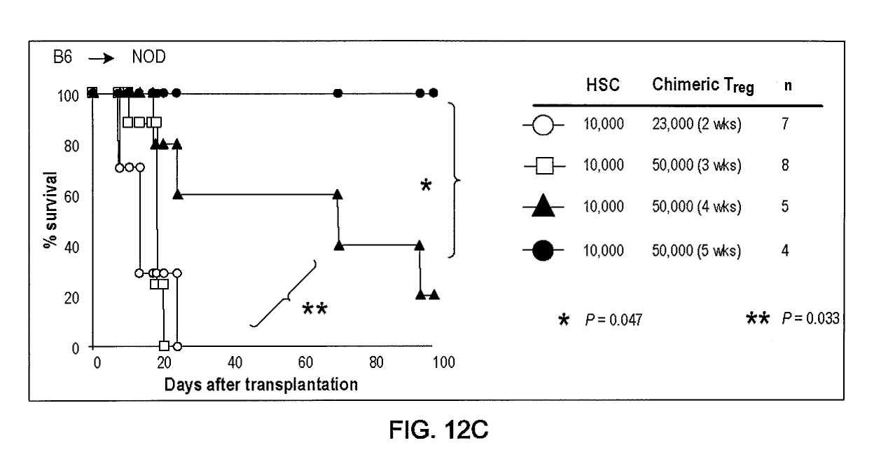

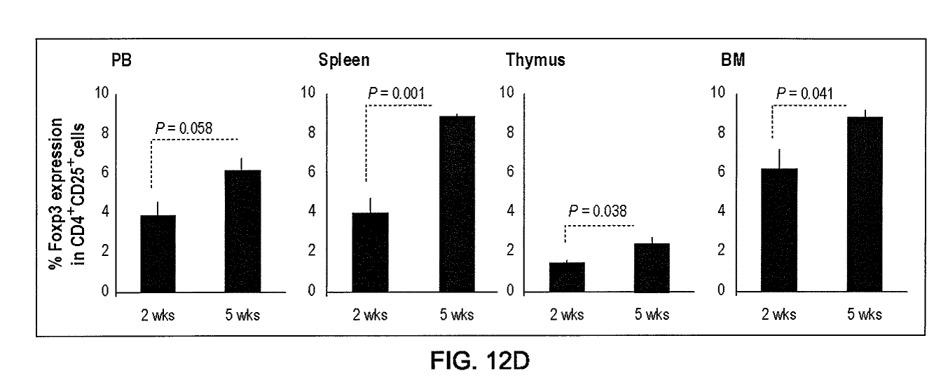

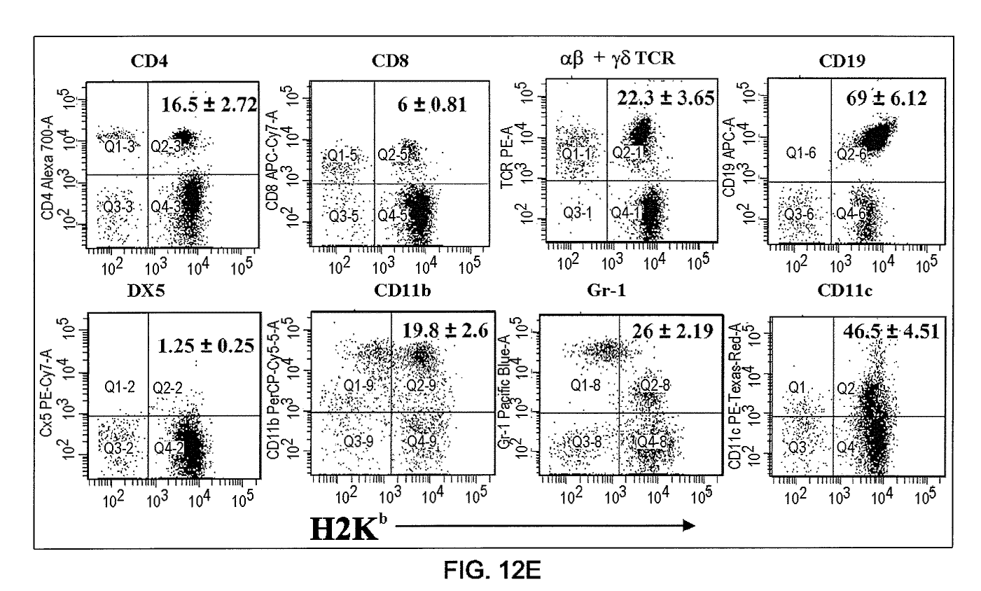

FIG. 12. Data demonstrating that chimeric T_{reg} potently enhanced HSC engraftment. CD8⁻/CD4⁺/CD25^{bright} T_{reg} were sorted from mixed chimeras (B6→NOD) spleens. Sorted 23,000 to 50,000 chimeric T_{reg} plus 10,000 B6 HSC were transplanted into ablatively conditioned NOD recipients. (A) Percent engraftment in NOD recipients of 10,000 B6 HSC+chimeric T_{reg}. (B) Percent donor chimerism in NOD mice. (C) Facilitative ability of chimeric T_{reg} administered to NOD mice. CD4⁻/CD25⁺ T_{reg} were sorted at selected time points: 2 week chimeric T_{reg} (♦), with 3 week chimeric T_{reg} (□), 4 week chimeric T_{reg} (▴), or 5 week chimeric T_{reg} (). (D) the level of Foxp3 expression in 2 week or 5 week chimeric T_{reg} of spleen, thymus, PB and bone marrow. (E) Multilineage PBL typing of NOD recipients of B6 HSC plus 5 week chimeric T_{reg}. The data are from one representative recipient 3 months after transplantation and analyzed based on the lymphoid and myeloid gate.

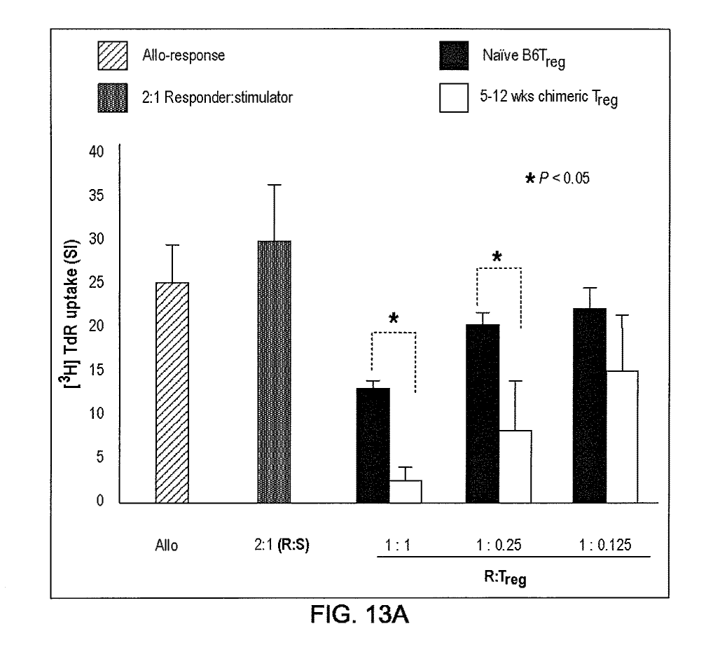

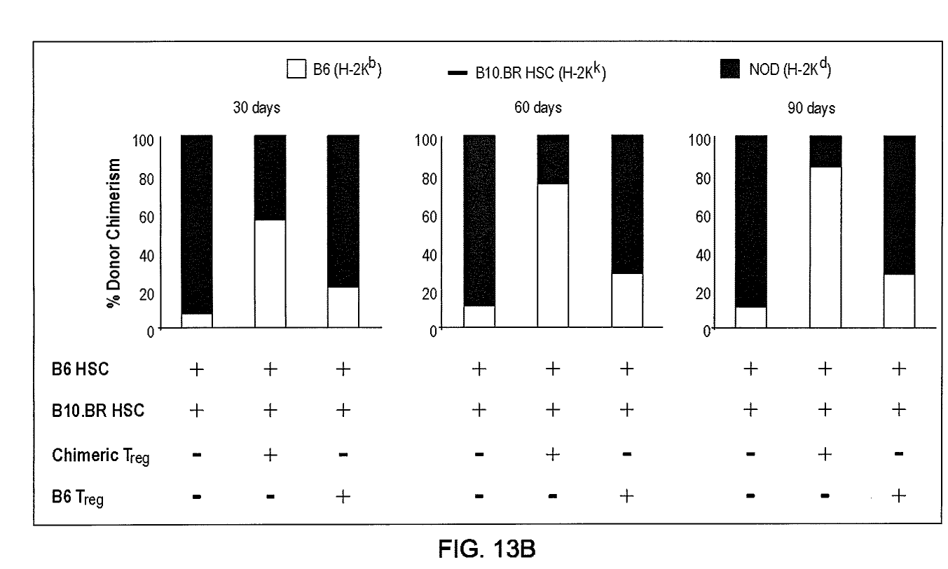

FIG. 13. The function of Chimeric T_{reg} is antigen-specific. (A) CD8⁻/CD4⁺/CD25^{bright} cells were sorted from the spleens of mixed chimeras (B6→NOD) or naïve B6 mice. Sorted T_{reg} were mixed with NOD lymphoid responder cells in decreasing ratios (1:1; 1:0.25; 1:0.125) and stimulated with irradiated B6 or NOD stimulator splenocytes. T cell proliferation was measured at 5 days. Results are Mean±SE of 3 to 4 independent experiments. (B) 10,000 B6 HSC and 10,000 B10.BR HSC with or without 100,000 sorted chimeric T_{reg} or naïve B6 T_{reg} were transplanted into NOD recipient mice conditioned with 950 cGy TBI. PBL typing was preformed by staining with anti-H-2K^{b}, H-2K^{k}, and H-2K^{d} mAbs at 30, 60, and 90 days. Analysis of donor (B6; □) or (B10.BR; −) origin and recipient (NOD; ▪) origin were based on lymphoid gate. Bar is mean.

### DETAILED DESCRIPTION

It is reported herein that facilitating cells (FCs) from NOD mice are functionally impaired. They fail to facilitate engraftment of syngeneic and allogeneic HSCs in vivo and do not enhance HSC clonogenicity in vitro. NOD FCs contain subpopulations similar to those previously described in B6 FCs, including p-preDCs, CD19⁺, NK1.1⁺DX5⁺ and myeloid cells. However, the CD19⁺ and NK1.1⁺DX5⁺ subpopulations are significantly decreased in number in NOD FCs compared to disease-resistant controls. Removal of the CD19⁺ or NK1.1⁻DX5⁺ subpopulations from FCs did not significantly affect facilitation. Notably, treatment of NOD donors with FLT3 ligand (FL) expanded the total number of FCs in peripheral blood and restored facilitating function in vivo.

These data demonstrate that NOD FCs exhibit significantly impaired function that is reversible, since FL restored production of functional FCs in NOD mice. These data suggest that FL plays an important role in the regulation and development of FC function, and that FCs may therefore be linked to diabetes pathogenesis and prevention.

Plasmacytoid Precursor Dendritic Cells (p-preDCs)

Plasmacytoid precursor dendritic cells (p-preDCs) have a phenotype of B220+CD11b−CD11c+ and is a precursor of dendritic cells. This cell type is found in lymphoid organs and expresses high levels of CD45RA, intermediate levels of CD11c, is a major producer of Type 1 interferon. These cells are also known as plasmacytoid T-cells. See, for example, O'Keeffe et al., “Mouse Plasmacytoid Cells: Long-lived Cells, Heterogeneous in Surface Phenotype and Function, that Differentiate Into CD8+ Dendritic Cells Only after Microbial Stimulus,” J. Exp. Med., 196(10):1307-1319 (2002).

### Antigen-Specific Regulatory T Cells (Tregs)

Regulatory T cells (Tregs) have the phenotype of CD4+/CD25+/FoxP3+, and have been referred to as “naturally-occurring” regulatory T cells. T regulatory cells are a component of the immune system that suppress immune responses of other cells, which is an important “self-check” built into the immune system. Tregs have been shown to play a role in the maintenance of self-tolerance; defects in Treg development or homeostasis result in systemic autoimmunity, while adoptive transfer of Treg as a therapeutic method can control ongoing autoimmune diseases. Recently, several studies have demonstrated a role for Treg in mediating transplantation tolerance in animal models.

### Methods of Screening Compounds

Methods are provided that can be used to identify compounds that stimulate the production of p-preDCs and/or Tregs in a sample of FCs. For example, the number of p-preDCs and/or Tregs in a sample of FCs in the presence of a compound can be compared to the number in a control sample. As used herein, “control sample” refers to FCs incubated in the absence of a compound but otherwise under the same or similar conditions as the FCs incubated in the presence of the compound. A compound is identified as stimulating the production of p-preDCs or Tregs if the number of p-preDCs or Tregs in the sample that was exposed to the compound is greater than the number in a control sample.

As used herein, a “compound” refers to, without limitation, a biological macromolecule, such as an oligonucleotide or a peptide, a chemical compound, a mixture of chemical compounds, or an extract isolated from bacterial, plant, fungal or animal matter.

The number of p-preDCs or Tregs can be determined using well known techniques in the art such as, without limitation, FACS. The cell-surface markers that can be used to identify such cells are disclosed below in the Examples. See, for example, Shapiro, Practical Flow Cytometry, 4^{th} Ed., Wiley-Liss, 2003.

### Methods of Treating Diabetes

Methods of treating an individual having diabetes are described herein. Methods of treating diabetes as described herein include administering a compound to the individual that increases the production of p-preDCs and/or Tregs. Compounds that can be administered can be a compound as identified herein that increases the number of p-preDCs and/or Tregs. Compounds determined to increase the number of p-preDCs and/or Tregs can be administered to individuals for the treatment of diabetes. For example, a compound deemed to increase the number of p-preDCs and/or Tregs can be administered to a patient having diabetes by any route of administration, including orally, nasally, intravenously, intraperitoneally, intramuscularly, subcutaneously, intrathecally, intradermally, intracisternally or intraventricularly.

The route of administration can depend on a variety of factors, such as treatment environment and therapeutic goals. A compound may be administered on a continuous or intermittent basis. For example, tablets or capsules can be prepared for oral administration by conventional means with pharmaceutically acceptable excipients, such as binding agents, fillers, lubricants, or wetting agents. In addition, liquid preparations for administration of a compound can take the form of, for example, solutions, syrups or suspensions. Such liquid preparations can be prepared by conventional means with pharmaceutically acceptable additives, such as suspending agents, emulsifying agents, non-aqueous vehicles, and preservatives. Liquid preparations can be presented as a dry product (e.g., for constitution with saline or other suitable liquid vehicle before administration) or in a nebulizer for nasal administration. Preparations can be suitably formulated to give controlled release of the compound.

Various pharmaceutically acceptable carriers can be used for in vivo administration of a compound to an individual, such as, for example, physiological saline or other known carriers appropriate to specific routes of administration. Preparations for administration can also contain buffer salts, flavoring, coloring and sweetening agents as appropriate. The dose of one or more compounds will depend on many factors, including the characteristics of the particular compound and the method and mode of administration. Typically, the concentration of a compound or compounds contained within a single dose will be an amount that effectuates a specific biological response without inducing significant or otherwise unacceptable levels of toxicity.

In one embodiment, the compound administered to a patient is a FLT3 ligand (FL) polypeptide. A representative sequence of a human FLT3 ligand is shown in SEQ ID NO:2, and the sequences of additional FLT3 ligands can be found in, for example, GenBank Accession Nos. AAA19825,AAA90949.1,AAI36465; NP_{—}001450.2,AAA90951.1, and AAA39436. A FLT3 ligand polypeptide also can be a polypeptide that has, for example, at least 90% sequence identity (e.g., at least 95% or at least 99% sequence identity) to SEQ ID NO:2.

(SEQ ID NO: 2)
MTVLAPAWSP TTYLLLLLLL SSGLSGTQDC SFQHSPISSD FAVKIRELSD YLLQDYPVTV
ASNLQDEELC GGLWRLVLAQ RWMERLKTVA GSKMQGLLER VNTEIHFVTK CAFQPPPSCL
RFVQTNISRL LQETSEQLVA LKPWITRQNF SRCLELQCQP DSSTLPPPWS PRPLEATAPT
APQPPLLLLL LLPVGLLLLA AAWCLHWQRT RRRTPRPGEQ VPPVPSPQDL LLVEH

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It will be appreciated that a single sequence can align differently with other sequences and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at ncbi.nlm.nih.gov on the World Wide Web. BLAST searches can be performed to determine percent sequence identity between a nucleic acid molecule and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence of the invention and another sequence, the default parameters of the respective programs are used.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### Examples

### Section A

### Example 1

### Mice

Four-to six-week-old NOD mice (male and female; Taconic Laboratories, Germantown, N.Y.), female nonobese resistant (NOR) mice, male C57BL/6 mice, and C57BL/10SnJ female mice (Jackson Laboratory, Bar Harbor, Me.) were used. Animals were housed in the barrier facility at the Institute for Cellular Therapeutics (Louisville, Ky.) and cared for according to National Institutes of Health animal care guidelines.

### Example 2

### Antibodies

All monoclonal antibodies (mAb) used in this study were purchased from BD Biosciences (San Diego, Calif.). c-Kit⁺Sca-1⁺Lin⁻ (HSC) sorting experiments used the following mAb: stem cell antigen-1 (Sca-1) phycoerythrin (PE), c-Kit allophycocyanin (APC), and the lineage panel consisting of: CD8α fluorescein isothiocyanate (FITC), Mac-1 FITC, B220 FITC, Gr-1 FITC, γδ-TCR FITC and β-TCR FITC. CD8⁺/TCR⁻ FC sorting experiments used β-TCR FITC, γδ-TCR FITC and CD8α PE. CD8⁺/TCR⁻/CD19⁻ sorting experiments used CD8α PE, β-TCR FITC, γδ-TCR FITC and CD19 APC. CD8⁺/TCR⁻/NK1.1⁻DX5⁻ cells were sorted by using CD8α APC, β-TCR FITC, γδ-TCR FITC, NK1.1 PE and DX5 PE.

### Example 3

### Sorting of HSC and FC

HSC and FC were isolated from BM by multiparameter, live sterile cell sorting (FACSVantage SE; Becton Dickinson, Mountainview, Calif.), as previously described (4). Briefly, BM was isolated and collected in a single cell suspension at a concentration of 100×10⁶ cells/ml in sterile cell sort media (CSM), containing sterile 1× Hank's Balanced Salt Solution without phenol red, 2% heat-inactivated fetal bovine serum, 10 mM/ml HEPES buffer, and 30 μl/ml gentamicin (GIBCO, Grand Island, N.Y.). Directly labeled mAbs were added at saturating concentrations and the cells were incubated for 30 min and washed with CSM.

### Example 4

### Phenotypic Analysis of Sorted CD8⁺/TCR⁻ FC

Sorted FC (purity was ≧95%) were incubated with Fc receptor block (anti-CD16/CD32) before staining with lineage-specific markers: anti-CD11 FITC, CD11b APC, CD14 FITC, NK1.1 FITC, DX5 FITC, B220 PerCP, Gr-1 APC and CD19 APC, as previously described (4). Sorted FC were analyzed for p-preDC FC as B220⁺/CD11c⁺/CD11b⁻ using Cell Quest Software (Becton Dickinson).

### Example 5

### DC Maturation and Cytokine Production

FC were cultured alone or with 1 μM TLR-9 ligand CpG-ODN 1668 (TCCATGACGTTCCGATGCT) (SEQ ID NO:1) (GIBCO BRL Custom Primers) (13) for 18 h. Supernatants were assayed for cytokines by Linco Diagnostic (St Charles, Mo.) using LINCOplex™ Multiplex immunoassay and cells were stained with anti-CD80, anti-CD86, or anti-MHC class II I-A^{d} (39-10-8) FITC mAb, with appropriately matched isotype controls.

### Example 6

### HSC and/or FC Transplantation

In the syngeneic model, recipients were conditioned with 950 cGy TBI from a Cesium source (Nordion, Ontario, ON, Canada) and transplanted with 500 HSC±30,000 FC populations by tail vein injection≧6 h after irradiation (14). In the allogeneic model, recipients conditioned with 950 cGy TBI were transplanted with 5,000 HSC±30,000 FL-PB FC (4).

### Example 7

### Colony-Forming Cell Assay

HSC were cultured at a 1:2 ratio with or without FC in methylcellulose containing mouse growth factors (MethoCult GF M3434; StemCell Technologies, Vancouver, BC, Canada) in duplicate at 37° C. in 5% CO₂ and humidified atmosphere (13). After 14 days, colonies containing more than 50 cells were scored.

### Example 8

### Chimerism Testing

Engraftment of donor cells was evaluated by PBL typing using 3-color flow cytometry, as previously described (15).

### Example 9

### FC Morphology

Wright-Giemsa staining was performed on cytospins of 100,000 FC after fixed in methanol. Slides were examined for dendritic morphology under optical microscopy.

### Example 10

### Statistical Analysis

Experimental data were evaluated for significant differences using the Student's test; P<0.05 was considered significant. Graft survival was calculated according to the Kaplan-Meier method (4).

### Example 11

### NOD FC Exhibit Specific and Significant Differences in Subpopulations Compared to Normal Controls

In normal mice, the CD8⁺/TCR⁻ FC (FC total) population is heterogeneous, with the dominant subpopulation phenotypically resembling p-preDC (B220⁺/CD11c⁺/CD11b⁻) (4). Smaller percentages of B cell (CD19⁺), NK cell (NK1.1⁺DX5⁺), granulocyte (Gr-1⁺), and monocyte (CD14⁺) subpopulations are also present in FC total from normal mice (4). We found that NOD and NOR FC are comprised of similar distinct heterogeneous subpopulations (FIGS. 1A and C), and showed a heterogeneous morphology with Wright-Giemsa staining under light microscopy (FIGS. 1B and D). P-preDC FC represent the major CD8⁺/TCR⁻ FC subpopulation in all strains (female and male NOD mice, female NOR mice, and male B6 mice) examined (FIG. 1E). The B220⁺/CD11c⁺ FC population in female and male mice NOD is significantly increased compared to control NOR or B6 mice (FIG. 1H; P<0.05). The B220⁻/CD11c⁺/CD11b⁺ subset is significantly decreased compared to NOR mice (FIG. 1F; P<0.007). As previously shown, the dominant cell population in CD19⁺ FC is pre-B cells (B220⁺/CD11c⁻/intracytoplasmic IgM⁺) (4). 14% of female NOD FC were CD19⁺, which is significantly decreased compared to NOR and B6 mice (FIG. 11, P<0.05). Approximately 0.27% of NOD FC are CD19⁺/CD11c⁺/B220⁺ cells (FIG. 1G), which is not significantly different compared with the control strain. DC with a similar phenotype from normal LN and spleen have been shown to function as p-preDC (16). Of the female NOD FC total, B220⁺/NK1.1⁺DX5⁺ and B220⁺/Gr-1⁺ populations were significantly decreased compared to B6 FC (FIGS. 1K and J). The B220⁺/CD14⁺ population was not significantly different in all strains examined (FIG. 1L).

### Example 12

### FC Produce Cytokines and Up-Regulate Activation Markers After Stimulation

We evaluated whether NOD FC resemble NOR FC in response to CpG-ODN stimulation. CD86 was up-regulated on NOR FC, while CD80 and class II expression was similar in the absence of CPG stimulation (FIGS. 2A and B). However, while CD86 was up-regulated on NOD FC, CD80 expression was markedly decreased with stimulation (FIGS. 2A and B). After CpG-ODN stimulation, the majority of NOR FC were in a more activated state compared to NOD FC, as evidenced by their dendritic morphology (FIG. 2C; right panel). In contrast, NOD FC did not exhibit a mature morphology after CpG treatment (FIG. 2C; left panel).

We also examined chemokine and cytokine production by NOD and NOR p-preDC FC after CpG-ODN stimulation. In the presence of CpG-ODN, p-preDC FC produced more MIP-1α/CCL3, RANTES/CCL5, IP-10, IL-6, and TNF-α, compared to the level of those in absence of stimulation (FIG. 2D). Notably, p-preDC FC from NOR mice produced higher amounts of IL-6 (5×), RANTES/CCL5 (3.5×), MIP-1α/CCL3 (2.1×), and TNF-α (1.9×) compared to NOD p-preDC FC (FIG. 2D). In addition, we found that NOR p-preDC FC produce GM-CSF more efficiently in response to CpG-ODN stimulation, while NOD p-preDC FC do not (FIG. 2D). Taken together, these data demonstrate that NOD p-preDC FC are impaired in ability to produce chemokines and cytokines after CpG-ODN stimulation.

### Example 13

### NOD CD8⁺/TCR⁻ FC Function is Significantly Impaired in vivo

We next examined the ability of NOD FC to facilitate HSC engraftment using syngeneic model (13,14). NOD recipients were ablatively conditioned with 950 cGy TBI and reconstituted with 500 HSC±30,000 FC sorted from NOD donors. Only 4 of 13 (31%) recipients of HSC plus FC and 4 of 17 (24%) recipients of HSC engrafted and survived up to 130 days (FIG. 3B). In striking contrast with normal controls (4), NOD FC did not improve HSC engraftment in NOD recipients, as evidenced by the similar engraftment of HSC with FC compared to the HSC alone (P=0.579).

We then examined the function of NOR FC. NOR mice are MHC-congenic to NOD mice, but do not develop diabetes (FIG. 3A) (5). Five (31%) of 16 recipients of HSC alone engrafted and survived up to 130 days. In contrast, 70% (7 of 10) recipients of HSC plus FC engrafted long-term with survival over 130 days (FIG. 3C). Therefore, NOR FC significantly enhance engraftment of HSC in limiting numbers of HSC (P=0.029).

To assess whether NOR FC facilitate engraftment of NOD HSC, 500 NOD HSC plus 30,000 NOR FC (n=15) were transplanted into NOD recipients conditioned with 950 cGy. All recipients of HSC alone expired before 130 days after transplantation (FIG. 3D). In striking contrast, the majority of (11 of 21) animals transplanted with NOD HSC and NOR FC survived over 130 days, demonstrating that NOR FC also facilitate engraftment of NOD HSC (FIG. 3D). As expected, NOD FC did not enhance engraftment of NOR HSC (n=16; FIG. 3E).

### Example 14

### NOD CD8⁺/TCR⁻ FC Failed to Promote Generation of Colonies from HSC

To evaluate the function of NOD FC in vitro, we tested them using the CFC assay, which enumerates the number of monolineage and multilineage colonies generated by HSC (13). NOR HSC co-cultured with NOR FC for 18 h, then cultured in methylcellulose for 14 days, generated significantly more colonies compared to NOR HSC alone (n=3; P=0.011; FIG. 4B). In contrast, NOD FC failed to enhance colony formation when cultured with NOD HSC (n=3; P=0.422; FIG. 4C). FIG. 4A shows representative appearance of CFC-GM and -GEMM for NOD HSC. FC alone did not generate colonies (FIGS. 4B and C).

### Example 15

### Removal of CD19⁻ or NK1.1⁻DX5⁺ Cells from FC Does Not Significantly Impair Facilitation

To define the function of CD19⁺ or NK1.1⁺DX5⁺ FC subpopulations, HSC, CD8⁺/TCR⁻ or CD8⁺/TCR⁻/CD19⁻ cells were sorted from NOR mice and tested in the syngeneic assay for in vivo facilitation (FIG. 5A). 44% (4 of 9) recipients of HSC plus CD8⁺/TCR⁻/CD19⁻ FC cells exhibited long-term engraftment and survived at least 110 days (FIG. 5B). 63% (5 of 8) animals given HSC+CD8⁻/TCR⁻ FC survived up to 110 days (FIG. 5B). There was no significant difference in survival between the HSC plus FC total group compared to the HSC plus FC from which CD19⁺ FC had been depleted (P=0.49). 23% ( 4/17) of recipients transplanted with HSC alone survived up to 110 days (FIG. 5B). These data suggest that the CD19⁺ subpopulation may not play an important role in facilitation, and therefore that the low numbers of these cells was not the cause of ineffective facilitation by NOD FC.

The contribution of the NK1.1⁺DX5⁺ FC subpopulation to total FC function was evaluated next. Donor NK cells have the potential to promote HSC engraftment and suppress GVHD in allogeneic transplantation (17). Our previous data showed that approximately 4-6% of FC are NK1.1⁺DX5⁺ cells (4). In NOD mice, 1-1.5% of FC express NK1.1⁺DX5⁺. To test the contribution of the NK1.1⁺DX5⁺ FC subpopulation to FC function, HSC, CD8⁺/TCR⁻ FC, and CD8⁺/TCR⁻/NK1.1⁻DX5⁻ cells were sorted from the marrow of B6 donors, 58% ( 7/12) recipients of HSC plus CD8⁺/TCR⁻ FC survived up to 110 days, while 42% ( 5/12) HSC plus CD8⁺/TCR⁻/NK1.1⁻DX5⁻ recipients survived over 110 days (FIG. 5C). Survival of both groups was significantly enhanced compared to the group that received HSC alone (P=0.009).

### Example 16

### FL-Mobilized NOD FC Facilitate HSC Engraftment in Allogeneic Recipients

It was previously reported that FL treatment of NOD mice restored production of defective mature myeloid DC, plasmacytoid DC in spleen and pancreatic lymph nodes, and significantly increased T_{reg} in pancreatic lymph nodes (18). This was associated with a significant delay in diabetes progression. To test whether FL-treatment can restore the function of NOD FC, the phenotype and function of FL-PB FCs were evaluated. NOD mice were treated with FL for 10 days. FC were sorted from PB, and sorted FC were stained with B220, CD11c, CD19, NK1.1DX5, and CD11b mAbs. There was a significant increase in B220⁻/CD11c⁺/CD11b⁺ DC and NK1.1⁺DX5⁺ subpopulations in FL-PB FC (FIG. 6A). The percentage of CD19⁺ FC and p-preDC FC remained at same levels as untreated NOD BM FC (FIG. 6A).

It was next evaluated whether FL-treatment can restore the facilitating function of NOD FC. FL mobilized NOD PB FC were in a more activated state than untreated NOD BM FC, as evidenced by their dendritic morphology (FIGS. 6B and 1B). To test function of FL-PB FC, HSC were sorted from BM of untreated NOD mice and FC from the PB of FL-treated NOD mice. Conditioned B10 recipients received 5,000 HSC plus 30,000 FL-PB FC. Control mice were transplanted with 5,000 HSC±30,000 FC from BM of untreated NOD mice. FL-PB FC significantly enhanced engraftment of HSC, as evidenced by 63% of recipients (n=8) who received HSC plus FL-PB FC survived 120 days (FIG. 6C). 13% and 20% of recipients of HSC alone (n=9) or HSC plus FC (n=8) from untreated NOD mice survived over 120 days, respectively (FIG. 6C).

To confirm that recipients of HSC plus FL-PB FC exhibited durable engraftment and multilineage reconstitution, animals were followed for >4 months. Three-color flow cytometric analysis was performed. Recipients of HSC alone showed the presence of cells of donor origin including DC (CD11c), macrophage (Mac-1) and granulocytes (Gr-1), NK cells (NK1.1DX5) and the presence of low levels of T cells (CD8, CD4, αβ-+γδ-TCR), and B cells (B220) (FIG. 6D). In contrast, recipients of HSC plus FL-PB FC showed donor chimerism for multilineages, including T cells, B cells, NK cells, macrophages, and granulocytes (FIG. 6E).

### Example 17

### Remarks

In the present study, the phenotype and function of NOD FC was evaluated. It is reported for the first time that NOD FC are functionally impaired in vivo and in vitro. As in disease-resistant controls, the B220⁺/CD11c⁺/CD11b⁻ p-preDC FC subpopulation represents the major subpopulation of CD8⁺/TCR⁻ FC in NOD BM. The CD19⁺ or NK1.1⁺DX5⁺ FC subpopulations were significantly decreased in NOD FC compared to those from B6 or MHC-congenic diabetes-resistant NOR mice (12). NOR FC significantly enhanced engraftment of NOR HSC. In striking contrast, NOD FC were completely impaired in function and did not facilitate HSC engraftment. Similarly, NOD FC were impaired in function in vitro. NOR p-preDC FC were more efficient at GM-CSF, IL-6, MIP-1α/CCL3, Rantes/CCL5, and TNF-α production in response to CpG compared to NOD p-preDC FC. Removal of the CD19⁺ or NK1.1⁻DX5⁺ FC subpopulations did not significantly impair facilitation. Notably, FL treatment of NOD mice expanded FC in peripheral blood (PB), and these FL-PB-FC significantly enhanced engraftment of HSC. The fact that FL-treatment restored the function of NOD FC suggests that FL may represent a key cytokine for the development and function of FC. FC may therefore be a critical link in diabetes pathogenesis and prevention and may provide a novel cell-based approach to restore self-tolerance and regulation in treatment of type 1 diabetes.

Without being bound by any particular mechanism, it is proposed that the defective function of NOD FC may be to an abnormal activation status of the p-preDC FC subpopulation or the presence of impaired function of a collaborative subpopulation in FC such as B cells or NK cells. This hypothesis offers an attractive explanation for the mechanism by which FC enhance HSC engraftment in vivo and induce tolerance.

It is shown here for the first time that FC from NOD mice exhibit a functional defect in facilitating HSC engraftment in vivo and impaired function in vitro as well. However, the fact that FL-treatment of NOD donors results in production of functional FC implies that the defect is probably not cell intrinsic, but rather due to a lacking signal or activated state. FL plays a critical role in the development of p-preDC in human and mice (29,30). The ability of FL to promote p-preDC development in vivo was confirmed by experiments showing that administration of FL into human volunteers led to an increase in the number of PB p-preDC in humans, and that FL transgenic mice have increased numbers of p-preDC, where FL deficient mice have less p-preDC (31). It has been shown that treatment of prediabetic NOD mice with FL significantly decreased insulitis and progression to diabetes and was associated with a significant increase in myeloid DC, plasmacytoid DC, and T_{reg} (18). When DC from NOD mice BM is treated with NF-κB-specific ODN in vitro, administration of DC into NOD mice can effectively prevent the onset of diabetes (32). FL is also a key cytokine for FC generation and expansion, as evidenced by FL-BM culture and the mobilization of FC in PB (4). FL-mobilized PB FC promote the establishment of donor chimerism and tolerance induction. In the present study, it was shown that FL treatment can restore the function of NOD FC, demonstrating that FL can promote that development and function of FC in NOD mice.

In NOD mice, the B cell subpopulation (CD19⁺) within total FC population is present at a much lower frequency compared to NOR and B6 controls. The function of the CD19⁺ FC subpopulation remains elusive. Removal of this subpopulation from normal donors did not impair facilitation. It is formally possible that the CD19⁺ FC subpopulation does not contribute to FC function or that there is redundancy in the system that is contributed from another FC collaborative subpopulations. NOD FC also contain significantly lower numbers of NK1.1⁺DX5⁺ cells compared with B6 or NOR mice. It was unclear whether the failure of FC function was due to decreased numbers of the NK FC subpopulation. NOD mice exhibit an abnormally low level of NK cell activity (7,41), and a defect in NK/T cells (42). To evaluate whether NK FC were involved in facilitation of HSC engraftment, allogeneic HSC transplantation (B6→C3H) was performed using FC depleted of the NK FC subpopulation. There was no difference in engraftment in mice that receive HSC plus FC total vs. FC depleted of NK FC, suggesting that NK FC did not contribute to facilitation.

Notably, it was found that NOD FC exhibit significantly impaired upregulation of CD86 following stimulation with CpG. Similarly, and in contrast with FC from diabetes-resistant donors, they failed to produce G-CSF and produced significantly lower levels of IL6 after CpG stimulation. Several groups have reported that NOD mice exhibited reduced T_{reg} frequency (48,49) and their impaired suppressive function has been linked to diabetes pathogenesis (50). The fact that wild-type FC can induce the generation of T_{reg}, but only in the presence of CpG-ODN (21), and that they are impaired in function in diabetes-prone NOD mice suggests that FC may also play a distinct role in diabetes pathogenesis.

In conclusion, the data reported herein reveal a novel defect in NOD FC function that is restored by treatment with FL. This data suggest a critical role of FL in development and maintaining the function of FC. These findings may have clinical implications for the treatment of type 1 diabetes and possibly other autoimmune disease states.

### Example 18

### References

1. Kaufman et al., Blood 84:2436-2446, 1994

2. Gandy et al., Immunity 11:579-590, 1999

3. Schuchert et al., Nature Medicine 6:904-909, 2000

4. Fugier-Vivier et al., J Exp Med 201:373-383, 2005

5. Anderson et al., Annu Rev Immunol 23:447-485, 2005

6. Serreze et al., J Immunol 150:2534-2543, 1993

7. Kataoka et al., Diabetes 32:247-253, 1983

8. Serreze et al., J Immunol 161:3912-3918, 1998

9. Cao et al., Curr Opin Immunol 19:24-30, 2007

10. Steptoe et al., J Immunol 168:5032-5041, 2002

11. Poligone et al., J Immunol 168:188-196, 2002

12. Prochazka et al., Diabetes 41:98-106, 1992

13. Rezzoug et al., J Immunol 180:49-57, 2008

14. Grimes et al., Exp Hematol 32:946-954, 2004

15. Huang et al., Blood 104:873-880, 2004

16. Munn et al., J Clin Invest 114:280-290, 2007

17. Ruggeri et al., Science 295:2097-2100, 2002

18. Chilton et al., Diabetes 53:1995-2002, 2004

19. Bridenbaugh et al., Blood 111:1735-1738, 2008

20. Colson et al., Blood 104:3829-3835, 2004

21. Taylor et al., J Immunol 179:2153-2162, 2007

22. Shatry et al., (Abstract). American Society of Hematology, 2007

23. Morelli et al., Nat Rev Immunol 7:610-621, 2007

24. Kawai et al., Nat Immunol 7:131-137, 2006

25. Abe et al., Am J Transplant 5:1808-1819, 2005

26. Pascual et al., Curr Opin Rheumatol 15:548-556, 2003

27. Gottenberg et al., Proc Natl Acad Sci USA 103:2770-2775, 2006

28. Greenberg et al., Ann Neurol 57:664-678, 2005

29. Gilliet et al., J Exp Med 195:953-958, 2002

30. Chen et al., Blood 103:2547-2553, 2004

31. Manfra et al., J Immunol 170:2843-2852, 2003

32. Ma et al., Diabetes 52:1976-1985, 2003

33. Jego et al., Curr Dir Autoimmun 8:124-139, 2005

34. Jego et al., Immunity 19:225-234, 2003

35. Eynon et al., J Exp Med 175:131-138, 1992

36. Yuschenkoff et al., J Immunol 157:1987-1995, 1996

37. Martin et al., Annu Rev Immunol 24:467-496, 2006

38. Raulet et al., Annu Rev Immunol 19:291-330.:291-330, 2001

39. Asai et al., J Clin Invest 101:1835-1842, 1998

40. Colonna et al., Nat Immunol 5:1219-1226, 2004

41. Ogasawara et al., Immunity 18:41-51, 2003

42. Wang et al., J Exp Med 194:313-320, 2001

43. Tang et al., Nat Immunol 7:551-553, 2006

44. Yamazaki et al., J Exp Med 198:235-247, 2003

45. Kared et al., Diabetes 54:78-84, 2005

46. Salomon et al., Immunity 12:431-440, 2000

47. Bour-Jordan et al., J Clin Invest 114:979-987, 2004

48. Pop et al., J Exp Med 201:1333-1346, 2005

49. Kukreja et al., J Clin Invest 109:131-140, 2002

50. Lindley et al., Diabetes 54:92-99, 2005

### Section B

### Example 1

### Mice

Four-to six-week-old NOD (H-2^{g}) female mice (Taconic Laboratories; Germantown, N.Y.), C57BL/6 (B6; H-2^{b}) and B10.BR (H-2^{k}) female mice (Jackson Laboratory; Bar Harbor, Me.) were used. Animals were housed in the barrier animal facility at the Institute for Cellular Therapeutics (Louisville, Ky.) and cared for according to National Institutes of Health animal care guidelines.

### Example 2

### Antibodies for Sorting

All monoclonal antibodies (mAbs) used in this study were purchased from PharMingen (San Diego, Calif.). HSC (c-Kit⁺/Sca-1⁺/Lin⁻; KSL) sorting experiments used the following mAb: stem cell antigen-1 (Sca-1) phycoerythrin (PE; E13-161.7; Rat IgG₂ₐ), c-Kit allophycocyanin (APC; 2B8; IgG_{2b}), and the lineage panel consisting of: CD8α fluorescein isothiocyanate (FITC; 53-6.7; Rat IgG₂ₐ), Mac-1 FITC (M1/70; IgG_{2b}), B220 FITC (RA3-6B2; Rat IgG₂ₐ), Gr-1 FITC (RB6-8C5; Rat IgG_{2b}), γδ-TCR FITC (GL3; Armenian hamster IgG) and β-TCR FITC (H57-597; Armenian hamster IgG). CD8⁺TCR⁻ FC sorting experiments used β-TCR FITC, γδ-TCR FITC and CD8α PE (53-6.7; IgG₂ₐ). P-pre DC FC were sorted by using β-TCR FITC, γδ-TCR FITC, CD8α APC (53-6.7; Rat IgG₂ₐ), CD11b FITC, CD11c PE (HL3; Armenian hamster IgG), and B220 APC-Cy7 (RA3-6B2; Rat IgG₂ₐ). CD8⁻CD4⁺CD25^{bright} T_{reg} were sorted by using CD4 APC (RM4-5; Rat IgG₂ₐ), CD25 PE (PC61; Rat IgG₁), and CD8α FITC.

### Example 3

### HSC, FC, and T_{reg} Sorting

HSC and FC were isolated from bone marrow by multiparameter, live sterile cell sorting (FACSVantage SE and FACSAria; Becton Dickinson, Mountainview, Calif.), as previously described (2). Briefly, bone marrow was isolated and resuspended in a single cell suspension at a concentration of 100×10⁶ cells/ml in sterile cell sort media (CSM), which contains sterile 1× Hank's Balanced Salt Solution without phenol red (GIBCO; Grand Island, N.Y.), 2% heat-inactivated fetal calf serum (FCS; GIBCO), 10 mM HEPES buffer (GIBCO), and 0.5% Gentamicin (GIBCO). Directly labeled mAb were added at saturating concentrations and the cells were incubated for 30 min on ice and washed twice. Cells were resuspended in CSM at 2.5×10⁶ cells/ml. CD8⁻/CD4⁺/CD25^{bright} T_{reg} were sorted from spleens of donor B6 or B6→NOD chimeric mice.

### Example 4

### HSC and/or FC T_{reg} Transplantation

In the HSC+FC allogeneic model, NOD recipients were conditioned with 950 cGy or 1050 cGy total body irradiation (TBI) from a Cesium source (Nordion, Ontario, Canada), and transplanted with 4,000, 5,000, or 10,000 B6 HSC with or without 30,000 FC or 45,000 B6 FC via lateral tail vein injection at least 6 hours after irradiation. The HSC and FC were mixed prior to injection. A group of irradiated untransplanted mice served as controls.

In the HSC+T_{reg} allogeneic model, CD8⁻/CD4⁺/CD25^{bright} T_{reg} were sorted from spleens of naïve B6 or B6→NOD chimeric mice. Various doses of T_{reg} plus B6 or B10.BR HSC were transplanted into NOD recipients conditioned with 950 cGy TBI.

### Example 5

### T_{reg} Generation in vivo

Recipient NOD mice were conditioned with 950 cGy of TBI and reconstituted with 1,000 syngeneic NOD HSC and 10,000 allogeneic B6 HSC with 45,000 CD8⁺/TCR⁻ FC or 45,000 FC without B220⁺/CD11c⁺/CD11b⁻ p-preDC by tail vein injection. Recipients were euthanized at 2, 3, 4, 5 weeks after transplantation. The thymus, spleen, and bone marrow were harvested, and donor (B6) and recipient (NOD) origin CD8⁻/CD4⁺/CD25^{bright}/FoxP3⁺ T_{reg} were analyzed by flow cytometry using Cell Quest Software (Becton Dickinson).

### Example 6

### Assessment of Chimerism

Donor engraftment in the recipients was evaluated by peripheral blood lymphocyte (PBL) typing using 4-color flow cytometry, as previously described (23). Briefly, whole blood from recipients was collected in heparinized tubes, and aliquots of 100 μl were stained with donor-specific anti-H-2K^{b} FITC (AF6-88.5; mouse IgG₂ₐ) along with a combination of the following mAbs (from PharMingen): CD8α PerCP (53-6.7; rat IgG₂ₐ), CD4 PerCP (RM4-5; rat IgG₂ₐ), β-TCR APC (H57-597; Armenian hamster IgG), Pan-NK cell PE (DX5; rat IgM), NK1.1 PE (PK136; mouse IgG₂ₐ), B220 PerCP (RA3-6B2; rat IgG₂ₐ), CD11c PE, Gr-1 PE (RB6-8C5; rat IgG_{2b}), and CD11b APC (M1/70; IgG_{2b}) mAbs.

### Example 7

### Mixed Lymphocyte Reaction and T_{reg} Suppression Assay in vitro

The in vitro suppression assay was carried out as previously described (24). Briefly, antigen presenting stimulator splenocytes were isolated from B6 and NOD stains. Cells were reconstituted in MLR media containing of DMEM with 5% fetal bovine serum, 1 mM sodium pyruvate, 2 mM glutamine, 100 U/ml penicillin, 100 U/ml streptomycin, 10 mM hepes, 0.05 mM 2-mercaptoethanol, 100 mM N-methyl-L-arginine, 0.5 mM L-arginine, 0.3 mM L-asparagine, 0.01 mM folic acid, and 1% NOD responder mouse serum. Splenocytes were then incubated overnight in a humidified chamber at 37° C. with 5% CO₂. 1×10⁵ lymphoid responder cells were isolated from naïve NOD animals, reconstituted in MLR media and then cultured with 1×10⁵ irradiated (2000 cGy) B6 or NOD stimulator splenocytes in triplicate in 96-well round-bottomed plates. Sorted CD8⁻/CD4⁺/CD25^{bright} T_{reg} from splenocytes of either mixed chimeras (B6→NOD) or naïve B6 mice were added to the stimulator/responder mix at 1:1, 1:0.25 and 1:0.125 responder/T_{reg} ratios for 4 days in a humidified chamber at 37° C. with 5% CO₂. The cell mix was pulsed on day 4 for an additional 18 hours with 10 μCi [³H] thymidine (Perkin Elmer, Boston, Mass.). The cell mix was harvested on the fifth day with an automated cell harvester (Tomtec Harvester 96; Wallac, Gaithersburg, Md.) and the radionucleotide uptake determined by scintillation counting (1205 BetaPlate, Wallac). The data are expressed as a stimulation index, determined from mean of triplicate determinations±standard error of the mean (SE). The ratio of counts per million generated by the host responder cells in response to a given stimulator relative to the auto-response of the host.

### Example 8

### Statistical Analysis

Experimental data were presented as the mean plus or minus SEM. Statistical significance was assessed using the student's t-test; P<0.05 was considered significant. Graft survival was calculated according to the Kaplan-Meier method (2).

### Example 9

### CD8⁺/TCR⁻ FC Enhanced Allogeneic B6 HSC Engraftment in NOD Recipients

It was previously reported that CD8⁺/TCR⁻ FC potently enhance engraftment of allogeneic HSC in diabetes-resistant recipients (1-3). Here, it was evaluated whether FC have a similar effect in prediabetic NOD recipient mice. NOD mice are relatively radioresistant, and require a higher bone marrow cell dose and higher levels of conditioning to establish allogeneic engraftment compared with wild-type mice (25). Therefore, titrations of HSC dose and TBI dose were carried out to establish the model. HSC (c-Kit⁺/Sca-1⁺/Lin⁻) were sorted from bone marrow of B6 donors, and 4,000, 5,000, or 10,000 HSC were transplanted into NOD recipients conditioned with 950 cGy or 1,050 cGy of TBI. In the 950 cGy TBI group, 0 (0%) of 5, 1 (11%) of 9 and 5 (19%) of 26 recipients of 4000, 5000 or 10,000 HSC engrafted, respectively (FIG. 7A). Only 20%-22% of recipients survived up to 100 days (FIG. 7C). In the 1050 cGy TBI group, 0 (0%) of 5, 0 (0%) of 9, or 5 (50%) of 10 recipients of 4,000, 5,000, or 10,000 HSC engrafted, respectively (FIG. 7A). Only 10% of recipients survived up to 100 days (FIG. 7D). The percent donor chimerism was not significantly different between the two groups that received 10,000 HSC (P=0.212; FIG. 7B).

Next, it was tested whether FC facilitate B6 HSC engraftment in allogeneic NOD recipient mice. HSC and CD8α⁺/TCR⁻ FC were sorted from B6 mice, and 10,000 HSC plus 30,000 or 45,000 FC were mixed and transplanted into NOD recipients conditioned with 950 cGy TBI. Only 19% ( 5/26) of recipients transplanted with HSC alone engrafted and survived up to 100 days (FIGS. 8A and 8C). Five of 18 (28%) recipients transplanted with HSC plus 30,000 FC engrafted (FIG. 8A) and survived up to 100 days (FIG. 8C). The percent donor chimerism in recipients of 30,000 FC+10,000 HSC was not significantly different compared with the group that received HSC alone (P=0.22; FIG. 8B). The survival of these mice was significantly longer than mice that received HSC alone (P=0.048; FIG. 8C). In contrast, 70% (7 of 10) of recipients given HSC plus 45,000 FC showed long-term engraftment, with survival over 100 days (FIGS. 8A and 8C). This was a significant difference from recipients of HSC alone (P=0.004). The percent donor chimerism in NOD recipients transplanted with HSC with 45,000 FC was significantly higher than recipients of HSC plus 30,000 FC or HSC alone (P=0.006; FIG. 8B). Previous studies showed that 30,000 FC are sufficient to significantly enhance engraftment of HSC in allogeneic diabetes-resistant recipients (1-3, 23). These data suggest that FC enhance engraftment of allogeneic HSC in NOD recipients, but higher numbers of FC are required compared to disease-resistant controls.

### Example 10

### FC Induce CD8⁻/CD4⁻/CD25^{bright}FoxP3⁺ T_{reg} in vivo

To determine whether FC-mediated facilitation of allogeneic HSC engraftment and tolerance occurs by induction of T_{reg} generation in vivo, the production of T_{reg} after HSC+FC transplantation was evaluated. CD8⁻/TCR⁻ FC were sorted from bone marrow of donor B6 mice and HSC from bone marrow of donor B6 and host NOD mice. 10,000 B6 HSC plus 1000 NOD HSC with or without 45,000 CD8⁺/TCR⁻ B6 FC were transplanted into recipient NOD mice conditioned with 950 cGy TBI in competitive repopulation assays (FIG. 9A). At 2, 3, 4, and 5 weeks after transplantation, thymus, spleen, and bone marrow were harvested from NOD recipients and the absolute numbers of donor (B6) or recipient (NOD) T_{reg} were determined by flow cytometry (FIG. 9B-D). As shown in FIG. 9E, donor- and recipient-derived CD4⁺/CD25⁺ FoxP3⁺ T_{reg} (chimeric T_{reg}) were detectable in thymus, spleen, and bone marrow at 2 weeks after transplantation. At two weeks, the highest members of T_{reg} were present in spleen and thymus, with absolute numbers increasing in PB, spleen, and bone marrow over time. The majority of T_{reg} were recipient-derived (89% to 97%). Only 3% to 11% of T_{reg} were donor-derived.

Previous studies have identified that the main subpopulation of FC is comprised of B220⁺/CD11c⁺/CD11b⁻ p-preDC FC (2, 26). P-preDC FC and pDC share many phenotypic, morphological, and functional features (2). P-preDC FC produce interferon (IFN)-α and tumor necrosis factor (TNF)-α in response to TLR-9 ligand (CpG-ODN) stimulation (2, 26). In addition, p-preDC FC express high levels of TLR9 (FIG. 10F).

To test whether the p-preDC subpopulation plays an important role in inducing T_{reg} production, 45,000 sorted B6 FC from which the B220⁻/CD11c⁺/CD11b⁻ p-preDC subpopulation had been removed were transplanted with 10,000 B6 HSC and 1,000 NOD HSC into conditioned NOD recipients. At 5 weeks after transplantation, thymus, spleen, and bone marrow were harvested from NOD recipients and the numbers of donor (B6) or recipient (NOD) T_{reg} were determined by flow cytometry. All animals (n=4) engrafted exclusively with only recipient HSC. They failed to engraft donor B6 HSC and did not produce chimeric T_{reg} (FIG. 10A-D). The absolute number of recipient-derived T_{reg} in PB, thymus, spleen, and BM was significantly decreased compared to mice that received FC TOTAL (FIG. 10E). These data suggest that the p-preDC subpopulation (CD8α⁺/B220⁺/CD11c⁺/CD11b⁻) in FC is a critical component in inducing chimeric T_{reg} generation in vivo.

### Example 11

### Chimeric T_{reg} Induced by FC Prevent Rejection and Potently Increase Long-Term Donor Chimerism

It has been shown that donor-derived CD4⁺/CD25⁺ T_{reg} inhibit lethal GVHD after allogeneic bone marrow transplantation (BMT) across major histocompatibility complex class I and II barriers in mice (23, 27, 28). FoxP3 is crucial in the development and function of natural CD4⁺/CD25⁺ T_{reg} (29-31). Significantly higher level of FoxP3 expression of in the CD4⁺/CD25^{bright} fraction compared to the CD4⁺/CD25^{dim} T_{reg} fraction (FIG. 11A) was observed. To investigate whether T_{reg} are involved in enhancement of engraftment of allogeneic HSC, the CD8⁻/CD4⁺/CD25^{bright} naïve T_{reg} function was tested in an allogeneic model for facilitation (B6→NOD). CD8⁻/CD4⁺/CD25^{bright} T_{reg} were sorted from spleens of naïve B6 mice. 10,000 B6 HSC plus 50,000, 100,000, or 200,000 T_{reg} were transplanted into NOD recipients conditioned with 950 cGy of TBI. Only 2 of 5 (40%) recipients of HSC plus 50,000 T_{reg} engrafted. Recipients exhibited low levels of donor chimerism (range: 0.5% -7.5%) and all survived less than 90 days (FIG. 11B-D). Six of eight (75%) recipients transplanted with HSC plus 100,000 T_{reg} engrafted (average % donor chimerism: 15%; range: 0.6%-82%) and 40% of the recipients survived up to 100 days (FIG. 11B-D). In contrast, 7 of 7 (100%) recipients of HSC+200,000 T_{reg} engrafted with high levels of donor chimerism (average: 60%; range: 1%-87%) and 71% survived over 100 days (FIG. 11B-D). These data suggest that naïve T_{reg} enhance engraftment of allogeneic HSC and this role is cell-dose dependent.

To evaluate the function of FC-induced chimeric T_{reg}, the ability of chimeric T_{reg} to enhance engraftment and donor chimerism following transplantation was tested. Spleens were harvested from mixed chimeras 2, 3, 4 and 5 weeks after HSC plus FC transplantation. CD8⁻/CD4⁺/CD25^{bright} chimeric T_{reg} were sorted from the spleens of chimeras and 50,000 chimeric T_{reg} plus 10,000 B6 HSC were transplanted into NOD recipients conditioned with 950 cGy of TBI. All secondary recipients of 2 week chimeric T_{reg} (n=7) or 3 week chimeric T_{reg} (n=8) plus HSC expired before 30 days after transplantation, suggesting that at these time points the T_{reg} are not functional (FIG. 12A-C). Three of 5 recipients of 4 week chimeric T_{reg} engrafted, with an average of 18% donor chimerism (range: 1.7%-79%; FIGS. 12A and B). Only 1 of 5 (20%) NOD mice receiving 4 week chimeric T_{reg} survived up to 100 days (FIG. 12C). In striking contrast, 100% of recipients (n=4) given 5 week chimeric T_{reg}+HSC engrafted and survived over 100 days (FIGS. 12A and C). All of the recipients showed donor cell chimerism in excess of 90% (range: 84%-95%; FIG. 12B). These data suggest that the FC-induced T_{reg} acquire function over at least 5 weeks.

The level of FoxP3 expression was recently reported to correlate with suppressive function of T_{reg} (29-31). The expression of FoxP3 was compared in 2 week vs. 5 week chimeric CD4⁺/CD25⁺ T_{reg} from mouse spleen, PB, thymus, and bone marrow (FIG. 12D). There was a significant increase in the level of FoxP3 expression in 5 week chimeric T_{reg} of spleen compared to 2 week chimeric T_{reg} (86.9±1.8 vs 38.9±7.6; p=0.001). There were also a significant increase in the level of FoxP3 expression in 5 week chimeric T_{reg} of thymus compared to 2 week chimeric T_{reg} (23.7±3.1 vs 14.1±1.8; p=0.038) and bone marrow (86.1±3.3 vs 61.0±9.1; p=0.041). However, there was no significant difference in 5 week chimeric T_{reg} of PB compared to 2 week chimeric T_{reg} (60.9±6.6 vs 38.5±6.9; p=0.058). These results indicate that FoxP3 gene expression is associated with the suppressive capacity of CD4⁺/CD25⁺ T_{reg} in vivo.

These mixed chimeras exhibited durable engraftment and showed the presence of multilineage donor cells including T cells (CD8, CD4, β-TCR), NK cells (NK1.1DX5), B cells (CD19), DC (CD11c), macrophage (Mac-1), and granulocytes (Gr-1) (FIG. 12E). These data suggest that 5 week chimeric T_{reg} are more efficient in suppressing immune responses and potently enhance engraftment of allogeneic B6 HSC in NOD recipient compared with naïve B6 T_{reg} (FIGS. 11B and D).

### Example 12

### Chimeric T_{reg} Potently Suppress Proliferation of T Cells in vitro

The suppressive function of chimeric T_{reg} was assessed in vitro by using MLR suppressor cell assays. CD8⁻/CD4⁺/CD25^{bright} T_{reg} were sorted from chimeric spleens 5 wks to 12 wks after HSC+FC transplantation. As shown in FIG. 13A, T_{reg} from naïve B6 mice resulted in 1.9 fold; 1.3 fold and 1.1 fold inhibition of proliferation at 1:1, 1:0.25, 1:0.125 responder/T_{reg} ratios (n=3). In contrast, chimeric T_{reg} potently suppressed T cell proliferation by 10.5 fold; 3.2 fold; and 1.7 fold at responder/T_{reg} ratios of 1:1, 1:0.25, 1:0.125 (n=4). Chimeric T_{reg} significantly suppressed T cell proliferation at responder/T_{reg} ratios of 1:1 and 1:0.25 compared with B6 T_{reg} (P<0.05). NOD responder splenocytes remained hypoproliferative in response to B6 stimulator and chimeric T_{reg} compared with stimulator plus B6 T_{reg}, suggesting that chimeric T_{reg} are significantly more potent than naïve B6 T_{reg} in suppressing effector T cell proliferation in vitro.

### Example 13

### Chimeric T_{reg} Enhance HSC Engraftment in an Antigen-Specific Fashion

It was next evaluated whether chimeric T_{reg} enhance engraftment of HSC in antigen-specific manner. Five week chimeric T_{reg} were sorted from spleens of mixed chimeras (B6→NOD). 100,000 chimeric T_{reg} were then mixed with 10,000 B6 HSC (donor specific)+10,000 B10.BR HSC (third party) and transplanted into irradiated NOD recipients. NOD mice given HSC plus B6 T_{reg} or HSC alone served as controls. Two of the four animals that received HSC alone engrafted and exhibited an average of 6.7% donor B6 chimerism at 30 days, 11.2% at 60 days, and 10.6% at 90 days (FIG. 13B). Three of five animals given HSC plus B6 T_{reg} engrafted with 21.3% donor B6 chimerism at 30 days, 28.8% at 60 days, and 28.9% at 90 days. In contrast, eight of nine mice recipients of HSC+chimeric T_{reg} engrafted with a high levels of donor B6 chimerism ranging from 56.3% at 30 days, 75.4% at 60 days to 85% at 90 days. None of the recipients exhibited engraftment of MHC-disparate third-party B10.BR HSC. These data show that chimeric T_{reg} enhance donor B6 HSC engraftment but not third-party B10.BR HSC, demonstrating that chimeric T_{reg} function in vivo in an antigen-specific fashion.

### Example 14

### Remarks

A major challenge to the clinical use of T_{reg} has been to obtain sufficient numbers of cells and to maintain their tolerogenic properties in vivo after in vitro expansion and transplantation (32). Most attempts at in vitro expansion have been limited by loss of regulatory function and FoxP3 expression. The present group was the first to discover CD8⁺/TCR⁻ graft FC, a novel cell population in bone marrow that potently enhances engraftment of HSC in both allogeneic (1) and syngeneic recipients (3). FC are heterogeneous, comprised of 60-69% CD11c⁺/CD11b⁻/B220⁺ p-preDC FC, 4-6% NK FC, 5% CD3ε⁺ FC, and 15% CD19⁺ FC (2). The plasmacytoid precursor dendritic cell (p-preDC) subpopulation in the FC population plays a critical role in facilitation (2). Removal of p-preDC FC completely abrogates facilitation of HSC in vivo. However, p-preDC FC do not replace FC TOTAL in function in vivo and in vitro (2, 33). FC prevent GVHD and uniquely remain tolerogenic after in vivo infusion (4). T_{reg} can be generated in vitro via co-culture with p-preDC FC (5). In this study, it was shown that FC induced the generation of CD4⁺/CD25⁺/FoxP3⁺ T_{reg} in mixed chimeras (B6→NOD). Although the majority of chimeric T_{reg} were recipient-derived, they exhibited antigen-specific function in vitro and in vivo that was acquired over 5 weeks post-transplantation. Chimeric T_{reg} are superior to naïve T_{reg} in suppressing the proliferation of effector T cells in vitro and their antigen-specificity is important in the enhancement of engraftment of allogeneic HSC in vivo. Notably, removal of p-preDC from FC TOTAL blocks their facilitation ability and prevents the in vivo generation of chimeric T_{reg}, suggesting that p-preDC FC play a critical role in T_{reg} generation in vivo.

Mature p-preDC activated by IL-3 plus CD40 ligand or by the TLR-9 ligand have been shown to upregulate the expression of inducible co-stimulator-ligand (ICOS-L) and the generation of IL-10 producing T_{reg} (34). Ochando et al. demonstrated that pDC as phagocytic antigen-presenting cells mediate tolerance to vascularized allografts by inducing T_{reg} development in vivo (20). Their data also demonstrated that the generation of T_{reg} depends on direct interaction between CD4⁺ T cells and pDC in lymph nodes of allograft recipients (20). A recent report demonstrated that liver pDC prevented oral T cell priming and induced systemic tolerance to CD4⁺ and CD8⁺ T cell-mediated delayed-type hypersensitivity (35). B220⁺/CD11c⁺/CD11b⁻ p-preDC FC display characteristic plasmacytoid morphology, low expression of MHC class II, CD80, and CD86, and produce interferon (IFN)-α, tumor necrosis factor-α and other cytokines in response to CpG-ODN (2, 26). P-preDC FC stimulated with CpG-ODN promote CD4⁺/CD25⁻ T cells differentiation into CD4⁺/CD25⁺/FoxP3⁺ T_{reg} cells in vitro (5). In the present studies, it was found that FC express toll-like receptor 9 (TLR9) and induce both the generation of donor and host-derived CD4⁺/CD25⁺/FoxP3⁺ T_{reg} (chimeric T_{reg}) in vivo. The majority of chimeric T_{reg} were recipient-derived. In contrast to naïve T_{reg}, in vivo FC-induced-chimeric T_{reg} potently enhance engraftment of allogeneic HSC in ablatively conditioned NOD recipients and are significantly more potent in suppressing T cell proliferation in MLR suppressor cells assays in vitro compared to naïve T_{reg}.

Several reports suggest that CD4⁺/CD25⁺/FoxP3⁺ T_{reg} are generated in the thymus (36-38), and FoxP3 is a critical regulator of their development and suppressive function (30). Evidence suggests that FoxP3⁺ T_{reg} can develop extrathymically under certain conditions (39-41). TBI is a part of the conditioning regimen for HSC transplantation. Mice receiving ablative irradiation exhibit severe thymic atrophy which results in peripheral T cell hypoplasia (42). The recovery of function of thymocytes in ablatively conditioned mouse irradiated recipients is 3-5 weeks after syngeneic BMT, while splenic function resumes 2-3 weeks later (43). A recent study showed that the recovery of functional donor-derived CD4⁺/CD25⁺/FoxP3⁺ T_{reg} occurred in recipient's thymus and lymph node 6 weeks after bone marrow transplantation (30). Our present findings show that chimeric T_{reg} are generated beginning at 2 weeks after HSC+FC transplantation but are not fully functional until 5 weeks after transplantation. These results support previous studies. In addition, FoxP3 expression has been shown to correlate directly with T_{reg} function. The level of FoxP3 expression in 5 week chimeric T_{reg} of spleen was significantly increased compared with 2 week and 3 week chimeric T_{reg} (P=0.001), suggesting that FoxP3 is essential for suppressive function of chimeric T_{reg}. These results provide evidence for FC-induced generation of chimeric T_{reg} in the thymic and splenic environments after FC:HSC transplantation in recipient animals.

Strategies for the production of antigen-specific T_{reg} for use in transplantation are being pursued. Most models have used in vitro expansion of cultured T_{reg}. However, a major limitation has been to identify an approach to achieve efficient expansion yet retain suppressive function. Joffre et al. reported that recipient CD4⁺/CD25⁺/FoxP3⁺ T_{reg} stimulated in vitro with alloantigens induced antigen-specific tolerance to bone marrow and subsequent skin and cardiac allografts (11). Another study found that in vitro expanded T_{reg} exhibit reduced levels of FoxP3 expression, which significantly impaired their immune suppressive function (44). Alternatively, the potent antigen-specific T_{reg} can be induced in vivo by targeting the antigens to dendritic cells under certain circumstances. In certain circumstances, maternal cells crossed the placenta and engrafted into human fetal tissues in utero, resulting in “maternal microchimerism,” and inducing the development of antigen-specific CD4⁺/CD25⁺/FoxP3⁺ T_{reg} (45). A recent study showed that donor-specific T_{reg} of recipient origin are recruited while donor antigens are present in low-intensity conditioning transplantation models and that these cells may play a critical role in the establishment of host-vs.-graft tolerance (46). It was found herein that FC-induced chimeric T_{reg} enhance donor-specific but not MHC-disparate third-party HSC engraftment in NOD recipients, suggesting that the function of chimeric T_{reg} is highly antigen-specific. The effect is very potent and ex vivo expansion of the FC population is not required. Most importantly, FC maintain their tolerogenic properties in vivo after transplantation. As such, FC may play a critical role in cell-based approaches for tolerance induction in vivo.

It is of note that pDC play important regulatory roles in allogeneic HSC and organ transplant outcome (47). A recent study showed that depletion of all pDC from bone marrow grafts resulted in an acceleration of mortality from GVHD while the depletion of mature pDC from G-CSF mobilized splenic grafts had no effect. These data suggest that donor bone marrow pDC, but not mature pDC, attenuate acute GVHD (48). A significantly higher ratio of pDC:mDC precursor cells in peripheral blood correlates with successful withdrawal of immunosuppression after liver transplantation (49). In addition, a high ratio of co-inhibitory programmed death ligand (PD-L)1 to costimulatory CD86 on circulating pDC is associated with elevated levels of T_{reg} in human liver transplant tolerance (50). It was previously demonstrated that FC facilitate engraftment of HSC in allogeneic recipients without causing GVHD (1, 2). Notably, removal of the p-preDC FC subpopulation completely abrogated facilitation. However, p-preDC FC and p-preDC did not replace CD8⁺/TCR⁻FC TOTAL in function. It has now been shown that removal of the p-preDC subpopulation from FC grafts resulted in significantly decreased frequencies of CD4⁺/CD25⁺ FoxP3⁻ T_{reg} in thymus, spleen, bone marrow and peripheral blood as compared with mice that received FC TOTAL. Moreover, the phenotypic T_{reg} that were generated in the FC from which the p-preDC subpopulation had been removed did not facilitate. These data provide further evidence that p-preDC play an important role in induction of T_{reg} generation in vivo.

Collectively, the first in vivo evidence of the role of FC in inducing antigen-specific chimeric T_{reg} is provided herein. Removal of p-preDC from FC failed to produce chimeric T_{reg}. The fact that the p-preDC subpopulation represents the majority of FC and plays a critical role in facilitation (2, 26) suggests that p-preDC FC could represent a key component in the induction of T_{reg} generation. These findings may provide a novel cell-based approach to induce tolerance and treat autoimmune disorders through immunomodulation and mixed chimerism.

### Example 15

### References

1. Kaufman et al., Blood, 1994, 84:2436-2446

2. Fugier et al., J Exp Med., 2005, 201:373-383

3. Grimes et al., Exp Hematol., 2004, 32:946-954

4. Colson et al., Blood, 2004, 104:3829-3835

5. Taylor et al., J Immunol., 2007, 179:2153-2162

6. Sakaguchi et al., Cell, 2008, 133:775-787

7. Sakaguchi et al., J Immunol., 1995, 155:1151-1164

8. Tang et al., J Exp Med., 2004, 199:1455-1465

9. Scalapino et al., J Immunol., 2006, 177:1451-1459

10. Mottet et al., J Immunol., 2003, 170:3939-3943

11. Joffre et al., Nat Med., 2008, 14:88-92

12. Hara et al., J Immunol., 2001, 166:3789-3796

13. Karim et al. J Immunol., 2004, 172:923-928

14. Golshayan et al., Blood, 2007, 109:827-835

15. Belkaid et al., Immunity, 2008, 29:362-371

16. Watanabe et al., Nature, 2005, 436:1181-1185

17. Cong et al., J Immunol., 2005, 174:2787-2795

18. Yamazaki et al., J Exp Med., 2003, 198:235-247

19. Huang et al., Stem Cells, 2006, 24:936-948

20. Ochando et al., Nat Immunol., 2006, 7:652-662

21. Abe et al, Am J Transplant, 2005, 5:1808-1819

22. Moseman et al., J Immunol., 2004, 173:4433-4442

23. Huang et al., Blood, 2004, 104:873-880

24. Colson et al., J Immunology, 1995, 155:4179-4188

25. Li et al., J Immunology, 1996, 156:380-388

26. Huang et al., Diabetes, 2008, 57:2360-2370

27. Hoffmann et al., J Exp Med., 2002, 196:389-399

28. Taylor et al., Blood, 2002, 99:3493-3499

29. Hori et al., Science, 2003, 299:1057-1061

30. Fontenot et al., Nat Immunol., 2003, 4:330-336

31. Zheng et al., Nat Immunol., 2007, 8:457-462

32. Buckner et al., Ann NY Acad Sci., 2008, 1143:151-169

33. Rezzoug et al., J Immunol., 2008, 180:49-57

34. Ito et al., J Exp Med., 2007, 204:105-115

35. Goubier et al., Immunity, 2008, 29:464-475

36. Liston et al., Proc Natl Acad Sci USA, 2008, 105:11903-11908

37. Fontenot et al., J Exp Med., 2005, 202:901-906

38. Jordan et al., Nat Immunol., 2001, 2:301-306

39. Bettelli et al., Nature, 2006, 441:235-238

40. Knoechel et al., J Exp Med., 2005, 202:1375-1386

41. Kretschmer et al., Nat Immunol., 2005, 6:1219-1227

42. Bolotin et al., Blood, 1996, 88:1887-1894

43. Cohn et al., Thymus, 1980, 2:61-74

44. Wan et al., Nature, 2007, 445:766-770

45. Mold et al., Science, 2008, 322:1562-1565

46. Weng et al., Proc Natl Acad Sci USA, 2007, 104:8415-8420

47. Solari et al., Transplantation, 2008, 85:1513-1522

48. Banovic et al., J Immunol., 2009, 182:912-920

49. Mazariegos et al., Am J Transplant, 2003, 3:689-696

50. Tokita et al., Transplantation, 2008, 85:369-377

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of screening for a compound that stimulates the production of p-preDC cells, comprising:
contacting facilitating cells (FCs) with a test compound; and
determining whether or not the test compound increases the number of p-preDC cells,
wherein an increase in the number of p-preDC cells is indicative of a compound that stimulates the production of p-preDC cells.

2. The method of claim 1, wherein the number of p-preDC cells is determined using FACS.

3. The method of claim 1, wherein the compound is selected from the group consisting of a polypeptide, a small molecule, and a chemical.

4. A method of screening for a compound that stimulates the production of Treg cells, comprising:
contacting facilitating cells (FCs) with a test compound; and
determining whether or not the test compound increases the number of Treg cells,
wherein an increase in the number of Treg cells is indicative of a compound that stimulates the production of Treg cells.

5. The method of claim 4, wherein the number of Treg cells is determined using FACS.

6. The method of claim 4, wherein the compound is selected from the group consisting of a polypeptide, a small molecule, and a chemical.

7. A method of treating an individual having diabetes, comprising:
administering a compound to said individual that increases the production of p-preDCs and/or Tregs in said individual.

8. The method of claim 7, wherein said compound is a polypeptide.

9. The method of claim 8, wherein said polypeptide has at least 90% sequence identity to the sequence shown in SEQ ID NO:2.

10. The method of claim 8, wherein said polypeptide has at least 95% sequence identity to the sequence shown in SEQ ID NO:2.

11. The method of claim 8, wherein said polypeptide has at least 99% sequence identity to the sequence shown in SEQ ID NO:2.

12. The method of claim 8, wherein said polypeptide has the sequence shown in SEQ ID NO:2.
